SEQUENCE LISTING (3 sequences)

SEQ ID NO: 1 - DNA, 3873 bp, Zea mays
  CDS (530)...(3560)
gtcgacccacgcgtccgccaacatccgcctcagattcccgtccatcgttcgtctccgctgtttcccgtccctcgtcctccgccgaaaccctagcagagctcctcctccgccttccctccggtcgccggagtgcctggtccggtcacgggggtggtttcgaggcctggattggcggagtgtttggaaggaggcaggggcgggacagtaggaatacataaattttctaggttttcagccgccctatgcctgacgcatccacgcgcccacgcctgtctccgcacacacgctgccacgcacaggcaccgctcgtccgctccagctcttgcgtcgcacggacagacacagctccaccctccatagctcctcttgccgctgcttgtgcgccggatcaccgcccccggcctgagtcttcggacagtgcgtccgcggcagagcatctgcggcctgccccctgcgggcggcggcgccagtgcggctcgccccctctggccgtcttggcctgattggaccgagaaccaggaggagggacatggaggggttcgccaagccgtccacgagctcggggtctggggtcacggttagggcctccgtcgccgcggcctccatagaggagaggttcgccgatctgtgcaaggccaagctgggactggacgagagcacgacgaggcaggcaatgcagctcttgaaggagaccaataacattctcaagtccagcatgtcttccttgggcggcggatcgcccgaggagatcgagaggttctggtctgcctgtgttctgtactgtgtgtcaaggctcagtaaagcggggaggtcgaaggaagatggcagtgtctcactttgccagattctgagggcatctaagctgaacattgtggattttttcaaggagatgccacagttctgcataaaggttgcacacattttgactggcctatatggttcagattgggaaaaaaggcttgagttgaaggaactgcaagcgaatgttgtccatttaagcttgctaagcagttactacaaacgtgcataccaggagctattcttgtcaaatgatggcaagagttcagacaattcttcagaatcaaataaccaagaggcttctgattattatcgctttggatggttgctctttttagtcctaagaatccaaacattcagcagattcaaggatcttgtgacatccacgaacgaactggtttctgtgttggctgtacttatcattcatgttcctgtgcgactaaggaatttcgatataaaggattcatcctactttggtaagaagtcagataggggagtcagtcttattgcttctctttgtgaaaaacatcatacctcagaagatgagttgagcaaagcactggagaagacaaatactctcataatggatattctgaaaaagaagccatgcccagctacttcagcatgccaacaggataatttgtctttcatcgatccagaaggcttgacagtttttaaggatttgcttcagggagactcattgaaaccaagtctgataatcctggaaaaggaatatgaaaatgcaattaacacaaaaggtgagctagatgagcgcatgtttgctaatgatgaggacagtttacttggtagtggaagcctttcaggtggagccattaatttaccaggcacaaagagaaagtatgatgttatggcctcacctgcaaaatcaatatcaagcccaaatccaatgtctcctccacgcttttgtctatctcctaaaggaaacggcttttgcaactcaaagatggctcctagcactccagtgagcacagccatgacaactgccaagtggcttcggaatacagtatctccccttccatcaagaccttctggggaactgttgcgcttcttctcggcctgtgacaaggatctaacagatgacattgcacacagagctgggattatacttggagccatattcacgagcagttcttttggtgaacgaatctgtactagtatgcgaagcgcaagtaggatggatgctatctggacagagcaaagaaaaatggaagctcttaagctatattatagggtcctcgaatctatgtgtagggcagagtcacagatcttgagtgggaacaatcttacatcacttttgtctaatgaacgtttccatcggtgtatgattgcatgttctgcggagttagttcttgcaactcacaagactgtcactatgatgttcccagctgtgctggagaagactggcattacagcatttgacttgagtaaagtcatagagggttttgttcggcatgaagatacactcccaagagagttaaagcgccatctgaattctcttgaggaacggcttctggaaagcatggcatgggagaaagggtcatcaatgtacaattccttaatcgttgcgaggccggcactgtctgtagaaataagccggttaggtttactggctgaaccaatgccatcgcttgatgcaattgcagcacatcataacatctcattgggtggcctgccacctcttcccttccagaaacaggagcgtttgcaagacaaagatgaagtcaggtctcccaaaagggcatgcactgagaggagaaacgtgctggtggacagcaattcattgcggtcaccagtgaaggatattattaaaccaaagttaccacctctccaatcagcttttgcaagtccaacaaggccgaatcctgcagcgggaggcgaaacgtgtgcagagacaggaataggcgtgttctttagcaagatatcgaagctcgcagccattagaatcagaagtctctgtgaaagactgcagcttccccagcaagttctggaacgggtgtattcccttgtgcagcaaatccttagccaacagactggccttttcttcaacaggcacattgaccagatcatactatgcagcatatatggagttgccaagatctctcaactggaactttcattcaaggagattattttcggctacaggaaacaacctcagtgcaagccacaagttttccgaagcgtctatgttcattggcccccaagaagccgtaacgggaaaacaggagaagaccacgtcgacatcatcactttctacaacgaagtattcattcctgccgtcaagtccttgttggtggaggtagggcctggcgccagcgccagtccaaagaagaaagaagaggaaaaaggtcctgctgatgtaggtccattcccagaatctccccggctggctcggttcccgaacctccccgacatgtctccaaagaaggtttctgctacccataacgtgtacgtctcaccgctacggtcatcgaagatggacactcttctgtccccgagctccaagagctactacgcgtgcgtcggcgagagcacctacgcgttccagagcccctccaaggacctgaaggctatcaacaaccgcctaaacagtgtgtccggcgggaagaaagtcagcggccggctgaacttcgacgtcgtcagcgatctggtcgtggccagtagcctcggcagtgaccgtgacgccaagcctgcggccgatcctgccaagacgacgccggtgaagtgcgagccatctgactcgtgatcgtcgtcgactctgatgaggcccttgttatagattgttatgtttcagatactcgtgagttagtagatggtaactagacgaggaggcccttgtttctgtttgcgtcttttctttttttgacgtgggaaacatctctgcgagcagctctacgttgtgtaaaatatatataaagaaagctgccccgatggctccccttaacccaactcgcgttgtagggagatgacaagcaccatggccatgtaacttcatagaggttttgagagagctgtgtaaatgcaagatggccatgtatcaacagcggatattttgtt

SEQ ID NO: 2 - protein, 1010 aa, Zea mays
MEGFAKPSTSSGSGVTVRASVAAASIEERFADLCKAKLGLDESTTRQAMQLLKETNNILKSSMSSLGGGSPEEIERFWSACVLYCVSRLSKAGRSKEDGSVSLCQILRASKLNIVDFFKEMPQFCIKVAHILTGLYGSDWEKRLELKELQANVVHLSLLSSYYKRAYQELFLSNDGKSSDNSSESNNQEASDYYRFGWLLFLVLRIQTFSRFKDLVTSTNELVSVLAVLIIHVPVRLRNFDIKDSSYFGKKSDRGVSLIASLCEKHHTSEDELSKALEKTNTLIMDILKKKPCPATSACQQDNLSFIDPEGLTVFKDLLQGDSLKPSLIILEKEYENAINTKGELDERMFANDEDSLLGSGSLSGGAINLPGTKRKYDVMASPAKSISSPNPMSPPRFCLSPKGNGFCNSKMAPSTPVSTAMTTAKWLRNTVSPLPSRPSGELLRFFSACDKDLTDDIAHRAGIILGAIFTSSSFGERICTSMRSASRMDAIWTEQRKMEALKLYYRVLESMCRAESQILSGNNLTSLLSNERFHRCMIACSAELVLATHKTVTMMFPAVLEKTGITAFDLSKVIEGFVRHEDTLPRELKRHLNSLEERLLESMAWEKGSSMYNSLIVARPALSVEISRLGLLAEPMPSLDAIAAHHNISLGGLPPLPFQKQERLQDKDEVRSPKRACTERRNVLVDSNSLRSPVKDIIKPKLPPLQSAFASPTRPNPAAGGETCAETGIGVFFSKISKLAAIRIRSLCERLQLPQQVLERVYSLVQQILSQQTGLFFNRHIDQIILCSIYGVAKISQLELSFKEIIFGYRKQPQCKPQVFRSVYVHWPPRSRNGKTGEDHVDIITFYNEVFIPAVKSLLVEVGPGASASPKKKEEEKGPADVGPFPESPRLARFPNLPDMSPKKVSATHNVYVSPLRSSKMDTLLSPSSKSYYACVGESTYAFQSPSKDLKAINNRLNSVSGGKKVSGRLNFDVVSDLVVASSLGSDRDAKPAADPAKTTPVKCEPSDS

SEQ ID NO: 3 - skipped

(19) United States
(12) Patent Application Publication
(10) Pub. No.: US 2003/0167505 A1
(43) Pub. Date: Sep. 4, 2003

(54) Cell cycle nucleic acids, polypeptides and uses thereof

(76) Inventors: Brian R. Dilkes, Tucson, AZ (US); William J. Gordon-Kamm, Urbandale, IA (US); Paolo Sabelli, Tucson, AZ (US); Brian A. Larkins, Tucson, AZ (US); Keith S. Lowe, Johnston, IA (US); Yuejin Sun, Westfield, IN (US); Chunsheng Zhang, North Charleston, SC (US); Yan Liu, Corona, NY (US)

Correspondence Address: PIONEER HI-BRED INTERNATIONAL INC., 7100 N.W. 62ND AVENUE, P.O. BOX 1000, JOHNSTON, IA, 50131, US

(21) Appl. No.: 10/094,778

(22) Filed: Mar. 11, 2002

Related U.S. Application Data
(60) Provisional application No. 60/276,541, filed on Mar. 16, 2001.

(51) Int. Cl. A01H 5/00; C07H 21/04; C12N 5/04

(52) U.S. Cl. 800/278; 435/419; 435/320.1; 536/23.6

(57) ABSTRACT

The invention provides isolated Rb3 nucleic acids and their encoded proteins that are involved in cell cycle regulation. The invention further provides recombinant expression cassettes, host cells, transgenic plants, and antibody compositions. The present invention provides methods and compositions relating to altering cell cycle protein content, cell cycle progression, cell number and/or composition of plants.

### CROSS REFERENCE TO RELATED APPLICATIONS

This application claims priority to U.S. Ser. No. 60/276,541 filed Mar. 16, 2001 the disclosure of which is herein incorporated by reference.

### TECHNICAL FIELD

The present invention relates generally to plant molecular biology. More specifically, it relates to nucleic acids and methods for modulating their expression in plants.

### BACKGROUND OF THE INVENTION

Cell division plays an important role during all phases of plant development. The continuation of organogenesis and growth responses to a changing environment requires precise spatial, temporal and developmental regulation of cell division activity in meristems (and in cells with the capability to form new meristems such as in lateral root formation). Such control of cell division is also important in organs themselves (i.e. separate from meristems per se), for example, in leaf expansion, secondary growth, and endoreduplication.

A complex network controls cell proliferation in eukaryotes. Various regulatory pathways communicate environmental constraints, such as nutrient availability, mitogenic signals such as growth factors or hormones, or developmental cues such as the transition from vegetative to reproductive stages. Ultimately, these regulatory pathways control the timing, frequency (rate), plane and position of cell divisions.

Plants have unique developmental features that distinguish them from other eukaryotes. Plant cells do not migrate, and thus only cell division, expansion and programmed cell death determine morphogenesis. Organs are formed throughout the entire life span of the plant from specialized regions called meristems. In addition, many differentiated cells have the potential to both dedifferentiate and to reenter the cell cycle. There are also numerous examples of plant cell types that undergo endoreduplication, a process involving nuclear multiplication without mitosis. The study of plant cell cycle control genes is expected to contribute to the understanding of these unique phenomena.

Current transformation technology provides an opportunity to engineer plants with desired traits. Major advances in plant transformation have occurred over the last few years. However, in many major crop plants, serious genotype limitations still exist. Transformation of some agronomically important crop plants continues to be both difficult and time-consuming. For example, it is difficult to obtain a culture response from some maize varieties. Typically, a suitable culture response has been obtained by optimizing medium components and/or explant material and source. This has led to success in some genotypes. While, transformation of model genotypes is efficient, the process of introgressing transgenes into production inbreds is laborious, expensive and time-consuming. It would save considerable time and money if genes could be introduced into and evaluated directly in commercial hybrids.

Current methods for genetic engineering in maize require a specific cell type as the recipient of new DNA. These cells are found in relatively undifferentiated, rapidly growing callus cells or on the scutellar surface of the immature embryo (which gives rise to callus). Irrespective of the delivery method currently used, DNA is introduced into literally thousands of cells, yet transformants are recovered at frequencies of 10⁻⁵ relative to transiently-expressing cells. Exacerbating this problem, the trauma that accompanies DNA introduction causes recipient cells to arrest the cell cycle, and accumulating evidence suggests that many of these cells are directed into apoptosis or programmed cell death. (Reference Bowen et al., Tucson International Mol. Biol. Meetings). Therefore it would be desirable to provide improved methods capable of increasing transformation efficiency in a number of cell types.

In spite of increases in yield and harvested area worldwide, it is predicted that over the next ten years, meeting the demand for corn will require an additional 20% increase over current production (Dowswell, C. R., Paliwal, R. L., Cantrell, R. P. (1996) Maize in the Third World, Westview Press, Boulder, Colo.).

The components most often associated with maize productivity are grain yield or whole-plant harvest for animal feed (in the forms of silage, fodder, or stover). Thus the relative growth of the vegetative or reproductive organs might be preferred, depending on the ultimate use of the crop. Whether the whole plant or the ear are harvested, overall yield will depend strongly on vigor and growth rate. It would therefore be valuable to develop new methods that contribute to the increase in crop yield.

### SUMMARY OF THE INVENTION

The invention provides isolated nucleic acids and their encoded proteins that are involved in cell cycle regulation. The invention further provides recombinant expression cassettes, host cells, transgenic plants, and antibody compositions. The present invention provides methods and compositions relating to altering cell cycle protein content, cell cycle progression, cell number and/or composition of plants.

In a further aspect, the present invention relates to a method of modulating gene transcription mediated by RB3 in a plant cell, comprising the steps of transforming a plant cell with an expression cassette comprising a nucleic acid of the present invention operably linked to a promoter, wherein the polynucleotide is in sense, antisense, or both orientations; the plant cell under cell-growing conditions; and expressing the nucleic acid for a time sufficient to modulate expression of the nucleic acids in the plant cell. Expression of the nucleic acid encoding the protein of the present invention can be increased or decreased relative to a nontransformed control plant.

In yet a further aspect, the present invention relates to a method of modulating higher order chromatin structure through interaction with heterochromatin-associated proteins in a plant cell, comprising the steps of transforming a plant cell with an expression cassette comprising a nucleic acid of the present invention operably linked to a promoter, wherein the polynucleotide is in sense, antisense, or both orientations; the plant cell under cell-growing conditions; and expressing the nucleic acid for a time sufficient to modulate expression of the nucleic acids in the plant cell. Expression of the nucleic acid encoding the protein of the present invention can be increased or decreased relative to a nontransformed control plant.

### DETAILED DESCRIPTION OF THE INVENTION

Sequence ID No. 1—maize Rb3 nucleotide sequence.

Sequence ID No. 2—maize RB3 polypeptide sequence.

Cell cycle transitions in multicellular eukaryotes are mediated by cyclin-dependent kinase (CDK) complexes (Nasmyth, 1993) that contain at least a catalytic subunit (the CDK) and a regulatory subunit (cyclins, which are specific for different phases of the cycle). At the G1 to S transition in the cell cycle, the Rb-E2F repressive complex blocks progress. In turn, the affinity of Rb for E2F is modulated by active CDK-cyclin complexes. Two such cyclins, D & E, when complexed with active CDKs participate in the phosphorylation of the Rb protein. The progressive phosphorylation of Rb relaxes its association with E2F, and the free E2F transcriptional factor activates expression of numerous DNA replication-associated genes.

In addition, Rb represses transcription through the recruitment of histone deacetylase or heterochomatin-associated proteins that mediate chromatin condensation. Progression through the cell cycle may be controlled through modulating the activity of E2F, histone deacetylases, or chromatin-associated proteins (for example, polycomb or chromo-domain proteins) by means of modulating RB3 expression and/or activity. Based upon results in other eukaryotes, RB3 expression and/or activity should block the G1/S transition and prevent cell division by binding the above proteins (E2F, histone deacetylases, or chromatin-associated proteins). Perturbation of the cell cycle through modulation of RB3 should result in increased transformation frequencies. Perturbation of the cell cycle through modulation of RB3 should also result in altered phenotypes such as changes in cell size, growth, and developmental programs.

The activity of RB3 protein refers to RB3 protein that will bind the transcriptional activator E2F, and in the process suppress progression from the G1 phase of the cell cycle into S-phase. Overexpression of Rb3 will increase the amount of active RB3 protein (increased RB3 activity) and thus increase binding of RB3 to E2F. Bound E2F is unable to activate transcription of replication-associated genes and the cell cycle does not progress into S-phase.

Conversely, if Rb3 transcription or translation is reduced (for example, through known antisense, hairpin, or cosuppression methods), the amount of active RB3 available to bind to E2F is reduced (decreased RB3 activity), and the free, active E2F activates DNA replication and progression through S-phase of the cell cycle. Activity of RB3 can be down-regulated by increasing the amount of proteins, peptides and/or other molecules that will interact with RB3 (referred to here as “interactors”) thus reducing RB3's capacity to bind E2F. The result of freeing up E2F will be stimulated cell cycle progression. An example of an interactor with RB3 that would reduce its activity would be to express a gene encoding an antibody raised specifically against RB3. Similarly, expression of a protein known to bind RB3 and reduce its cell cycle suppressive activity would constitute an example of an interactor. Expression of Wheat Dwarf Virus RepA (or a similar geminiviral RepA) or an active peptide fragment thereof, could be used in this fashion. Another potential interactor would be pharmaceuticals, derived through screening assays based on their ability to bind and inactivate RB3.

Modulation of the cell cycle by altering activity of RB3 in the cell can take various forms, including either stimulating or suppressing the cell cycle and thus cell division. Likewise, endoreduplication can be stimulated or suppressed, depending on how RB3 activity is affected.

A reduction of RB3 activity which will stimulate cell division could be envisaged in any cell type in the plant, including those of the root, stem, leaves, flowers, and developing fruits and seed. However, a preferred target cell for stimulating cell division would be cells known to have a capacity for division, such as cells of the root apical meristem, the root quiescent center, pericycle cells, the apical meristem, secondary or axillary meristems, developing influorescenses, scutellar cells, cambial cells, and the like.

A reduction of RB3 activity leading to increased levels of endoreduplication could be envisaged in any plant cell type, but preferred targets might be cells either predisposed to this process or actively undergoing the process (but to an extent where endoreduplication could be further stimulated). This would include such cell types as endosperm and various leaf cell types. Conversely, increasing RB3 activity would reduce cell division and/or endoreduplication in the various cell types listed above.

### DEFINITIONS

The term “isolated” refers to material, such as a nucleic acid or a protein, which is: (1) substantially or essentially free from components that normally accompany or interact with it as found in its natural environment. The isolated material optionally comprises material not found with the material in its natural environment; or (2) if the material is in its natural environment, the material has been synthetically altered or synthetically produced by deliberate human intervention and/or placed at a different location within the cell. The synthetic alteration or creation of the material can be performed on the material within or apart from its natural state. For example, a naturally-occurring nucleic acid becomes an isolated nucleic acid if it is altered or produced by non-natural, synthetic methods, or if it is transcribed from DNA which has been altered or produced by non-natural, synthetic methods. The isolated nucleic acid may also be produced by the synthetic re-arrangement (“shuffling”) of a part or parts of one or more allelic forms of the gene of interest. Likewise, a naturally-occurring nucleic acid (e.g., a promoter) becomes isolated if it is introduced into a different locus of the genome. Nucleic acids which are “isolated,” as defined herein, are also referred to as “heterologous” nucleic acids.

As used herein, “polypeptide” means proteins, protein fragments, modified proteins, amino acid sequences and synthetic amino acid sequences. The polypeptide can be glycosylated or not.

As used herein, “plant” includes but is not limited to plant cells, plant tissue, plant organs, plant pieces and plant seeds.

As used herein, “promoter” includes reference to a region of DNA upstream from the start of transcription and involved in recognition and binding of RNA polymerase and other proteins to initiate transcription.

By “fragment” is intended a portion of the nucleotide sequence or a portion of the amino acid sequence and hence protein encoded thereby. Typically, fragments of a nucleotide sequence may encode protein fragments that retain the biological activity of the native nucleic acid. However, fragments of a nucleotide sequence, which are useful as hybridization probes, generally do not encode fragment proteins retaining biological activity. Fragments of a nucleotide sequence are generally greater than least 25, 50, 75, 100, 125, 150, 200, 250, 300, 350, 400, or 500 nucleotides and up to the entire nucleotide sequence encoding the proteins of the invention. Generally, probes are less than 1000 nucleotides and preferably less than 500 nucleotides. Fragments of the invention include antisense sequences used to decrease expression of the inventive nucleic acids. Such antisense fragments may vary in length ranging from at least about 20 nucleotides, about 50 nucleotides, about 100 nucleotides, up to and including the entire coding sequence.

By “suppression” is intended reduction in expression of a cellular gene product.

By “functional fragment” is intended a portion of the nucleotide sequence or a portion of the amino acid sequence and hence protein encoded thereby, characterized by their ability, upon introduction to cells, to affect the G1 to S-phase transition. A functional fragment of a cell cycle gene, such as CDK, cyclin D or E2F, is manifested by increased DNA replication in a population of cells and by increased cell division rates. A functional fragment of a cell cycle repressor gene, such as Rb3, is manifested by decreased DNA replication in a population of cells and by decreased cell division rates. A functional fragment also includes a portion of a polynucleotide which suppresses the expression of the inventive polynucleotides.

By “variants” is intended substantially similar sequences. Generally, nucleic acid sequence variants of the invention will have at least 60%, 65%, 70%, 75%, 80%, 85%, 90%, 92%, 94%, 96%, 98%, or 99% sequence identity to the native nucleotide sequence. Generally, polypeptide sequence variants of the invention will have at least 60%, 65%, 70%, 75%, 80%, 85%, 90%, 92%, 94%, 96%, 98%, or 99% sequence identity to the native protein.

As used herein, “sequence identity” or “identity” in the context of two nucleic acid or polypeptide sequences includes reference to the residues in the two sequences that are the same when aligned for maximum correspondence over a specified comparison window, for example the entire coding sequence. An indication that two peptide sequences are substantially identical is that one peptide is immunologically reactive with antibodies raised against the second peptide. A polypeptide is substantially identical to a second polypeptide, for example, where the two polypeptides differ only by a conservative substitution. As used herein, sequence identity is determined using the GCG/bestfit program, GAP 10 using a gap creation penalty of 50 and a gap extension penalty of 3.

GAP uses the algorithm of Needleman and Wunsch (J. Mol. Biol. 48:443-453, 1970) to find the alignment of two complete sequences that maximizes the number of matches and minimizes the number of gaps. Default gap creation penalty values and gap extension penalty values in Version 10 of the Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater. The scoring matrix used in Version 10 of the Wisconsin Genetics Software Package is BLOSUM62 (see Henikoff & Henikoff (1989) Proc. Natl. Acad. Sci. USA 89:10915).

By “functionally equivalent” is intended that the sequence of the variant defines a chain that produces a protein having substantially the same biological effect as the native protein of interest. The variant is catalytically active.

By “modulate” is intended to increase, decrease, influence or change.

By “catalytically active” is intended the ability of a protein to bind to retinoblastoma (Rb) or is involved in stimulating DNA replication during the cell cycle.

By “interactor” is intended genes, proteins, polypeptide fragments, antibodies, pharmaceuticals, chemicals, aptamers and peptides capable of modulating expression or activity.

### NUCLEIC ACIDS

The isolated nucleic acids of the present invention can be made using (a) standard recombinant methods, (b) synthetic techniques, or combinations thereof. In some embodiments, the polynucleotides of the present invention will be cloned, amplified, or otherwise constructed from a monocot or dicot plant. Typical monocots include corn, sorghum, barley, wheat, millet, or rice. Typical dicots include soybean, sunflower, canola, alfalfa, cotton, potato, oil-seed Brassica or cassava.

Functional fragments included in the invention can be obtained using primers that selectively hybridize under stringent conditions. Fragments can be made through site-directed mutagenesis, restriction, change, DNA shuffling or a variety of methods known in the art. Primers are generally at least 12 bases in length and can be as long as 200 bases, but will generally be from 15 to 75, or from 15 to 50. Functional fragments can be identified using a variety of techniques such as restriction analysis, Southern analysis, primer extension analysis, and DNA sequence analysis and then tested for catalytic activity.

The present invention includes a plurality of polynucleotides that encode for the identical amino acid sequence. The degeneracy of the genetic code allows for such “silent variations” which can be used, for example, to selectively hybridize and detect allelic variants of polynucleotides of the present invention. Additionally, the present invention includes isolated nucleic acids comprising allelic variants. The term “allele” as used herein refers to a related nucleic acid of the same gene.

Variants of nucleic acids included in the invention can be obtained, for example, by oligonucleotide-directed mutagenesis, linker-scanning mutagenesis, mutagenesis using the polymerase chain reaction, and the like. See, for example, Ausubel, pages 8.0.3-8.5.9. Also, see generally, McPherson (ed.), DIRECTED MUTAGENESIS: A Practical Approach, (IRL Press, 1991). Thus, the present invention also encompasses DNA molecules comprising nucleotide sequences that have substantial sequence similarity with the inventive sequences.

Variants included in the invention may contain individual substitutions, deletions or additions to the nucleic acid or polypeptide sequences. Such changes will alter, add or delete a single amino acid or a small percentage of amino acids in the encoded sequence. Variants are referred to as “conservatively-modified variants” where the alteration results in the substitution of an amino acid with a chemically similar amino acid. When the nucleic acid is prepared or altered synthetically, advantage can be taken of known codon preferences of the intended host.

The present invention also includes “shufflents” produced by sequence shuffling of the inventive polynucleotides to obtain a desired characteristic. Sequence shuffling is described in PCT publication No. 96/19256. See also, Zhang, J. -H., et al. Proc. Natl. Acad. Sci. USA 94:4504-4509 (1997).

The present invention also includes the use of 5′ and/or 3′ UTR (untranslated regions) for modulation of translation of heterologous coding sequences. Positive sequence motifs include translational initiation consensus sequences [(Kozak, Nucleic Acids Res. 15:8125 (1987)] and the 7-methylguanosine cap structure [(Drummond et al., Nucleic Acids Res. 13:7375 (1985)]. Negative elements include stable intramolecular 5′ UTR stem-loop structures [(Muesing et al., Cell 48:691 (1987)] and AUG sequences or short open reading frames preceded by an appropriate AUG in the 5′ UTR [(Kozak, supra; Rao et al., Mol. and Cell. Biol. 8:284 (1988)].

Further, the polypeptide-encoding segments of the polynucleotides of the present invention can be modified to alter codon usage. Altered codon usage can be employed to alter translational efficiency and/or to optimize the coding sequence for expression in a desired host or to optimize the codon usage in a heterologous sequence for expression in maize. Codon usage in the coding regions of the polynucleotides of the present invention can be analyzed statistically using commercially available software packages such as “Codon Preference” available from the University of Wisconsin Genetics Computer Group [(see Devereaux et al., Nucleic Acids Res. 12:387-395 (1984)] or MacVector 4.1 (Eastman Kodak Co., New Haven, Conn.).

ESTs (Expressed Sequence Tags) encoding RB3 can be identified by conducting BLAST [(Basic Local Alignment Search Tool; Altschul, S. F., et al., (1993) J. Mol. Biol. 215:403410; see also www.ncbi.nlm.nih.gov/BLAST/)] searches for similarity to sequences contained in the BLAST “nr” database (comprising all non-redundant GenBank CDS translations, sequences derived from the 3-dimensional structure

Brookhaven Protein Data Bank:, the last major release of the SWISS-PROT protein sequence database, EMBL, and DDBJ databases). The cDNA sequences can be analyzed for similarity to all publicly available DNA sequences contained in the “nr,′ database using the BLASTN algorithm provided by the National Center for Biotechnology Information (NCBI). The DNA sequences can be translated in all reading frames and compared for similarity to all publicly available protein sequences contained in the “nr” database using the BLASTX algorithm [(Gish, W. and States, D. J. (1993) Nature Genetics 3:266-272)] provided by the NCBI. For convenience, the P-value (probability) of observing a match of a cDNA sequence to a sequence contained in the searched databases merely by chance as calculated by BLAST are reported herein as “pLog” values, which represent the negative of the logarithm of the reported P-value. Accordingly, the greater the pLog value, the greater the likelihood that the cDNA sequence and the BLAST “hit” represent homologous proteins.

For example, the inventive nucleic acids can be optimized for enhanced or suppressed expression in organisms of interest. See, for example, EPA0359472; WO91/16432; Perlak et al. (1991) Proc. Natl. Acad. Sci. USA 88:3324-3328; and Murray et al. (1989) Nucleic Acids Res. 17:477-498. In this manner, the genes can be synthesized utilizing species-preferred codons. See, for example, Murray et al. (1989) Nucleic Acids Res. 17:477-498, the disclosure of which is incorporated herein by reference.

The present invention provides subsequences comprising isolated nucleic acids containing at least 16 contiguous bases of the inventive sequences. For example the isolated nucleic acid includes those comprising at least 20, 25, 30, 40, 50, 60, 75 or 100 contiguous nucleotides of the inventive sequences. Subsequences of the isolated nucleic acid can be used to modulate or detect gene expression by introducing into the subsequences compounds which bind, intercalate, cleave and/or crosslink to nucleic acids.

The nucleic acids of the invention may conveniently comprise a multi-cloning site comprising one or more endonuclease restriction sites inserted into the nucleic acid to aid in isolation of the polynucleotide. Also, translatable sequences may be inserted to aid in the isolation of the translated polynucleotide of the present invention. For example, a hexa-histidine marker sequence provides a convenient means to purify the proteins of the present invention.

A polynucleotide of the present invention can be attached to a vector, adapter, promoter, transit peptide or linker for cloning and/or expression of a polynucleotide of the present invention. Additional sequences may be added to such cloning and/or expression sequences to optimize their function in cloning and/or expression, to aid in isolation of the polynucleotide, or to improve the introduction of the polynucleotide into a cell. Use of cloning vectors, expression vectors, adapters, and linkers is well known and extensively described in the art. For a description of such nucleic acids see, for example, Stratagene Cloning Systems, Catalogs 1995, 1996, 1997 (La Jolla, Calif.); and, Amersham Life Sciences, Inc, Catalog '97 (Arlington Heights, Ill.).

The isolated nucleic acid compositions of this invention, such as RNA, cDNA, genomic DNA, or a hybrid thereof, can be obtained from plant biological sources using any number of cloning methodologies known to those of skill in the art. In some embodiments, oligonucleotide probes that selectively hybridize, under stringent conditions, to the polynucleotides of the present invention are used to identify the desired sequence in a cDNA or genomic DNA library.

Exemplary total RNA and mRNA isolation protocols are described in Plant Molecular Biology: A Laboratory Manual, Clark, Ed., Springer-Verlag, Berlin (1997); and, Current Protocols in Molecular Biology, Ausubel et al., Eds., Greene Publishing and Wiley-Interscience, New York (1995). Total RNA and mRNA isolation kits are commercially available from vendors such as Stratagene (La Jolla, Calif.), Clontech (Palo Alto, Calif.), Pharmacia (Piscataway, N.J.), and 5′-3′ (Paoli, Pa.). See also, U.S. Pat. Nos. 5,614,391; and, 5,459,253.

Typical cDNA synthesis protocols are well known to the skilled artisan and are described in such standard references as: Plant Molecular Biology: A Laboratory Manual, Clark, Ed., Springer-Verlag, Berlin (1997); and, Current Protocols in Molecular Biology, Ausubel et al., Eds., Greene Publishing and Wiley-lnterscience, New York (1995). cDNA synthesis kits are available from a variety of commercial vendors such as Stratagene or Pharmacia.

An exemplary method of constructing a greater than 95% pure full-length cDNA library is described by Carninci et al., Genomics 37:327-336 (1996). Other methods for producing full-length libraries are known in the art. See, e.g., Edery et al., Mol. Cell Biol. 15(6):3363-3371 (1995); and, PCT Application WO 96/34981.

It is often convenient to normalize a cDNA library to create a library in which each clone is more equally represented. A number of approaches to normalize cDNA libraries are known in the art. Construction of normalized libraries is described in Ko, Nucleic Acids Res. 18(19):5705-5711 (1990); Patanjali et al., Proc. Natl. Acad. U.S.A. 88:1943-1947 (1991); U.S. Pat. Nos. 5,482,685 and 5,637,685; and Soares et al., Proc. Natl. Acad. Sci. USA 91:9228-9232 (1994).

Subtracted cDNA libraries are another means to increase the proportion of less abundant cDNA species. See, Foote et al. in, Plant Molecular Biology: A Laboratory Manual, Clark, Ed., Springer-Verlag, Berlin (1997); Kho and Zarbl, Technique 3(2):58-63 (1991); Sive and St. John, Nucleic Acids Res. 16(22):10937 (1988); Current Protocols in Molecular Biology, Ausubel et al., Eds., Greene Publishing and Wiley-lnterscience, New York (1995); and, Swaroop et al., Nucleic Acids Res. 19(8):1954 (1991). cDNA subtraction kits are commercially available. See, e.g., PCR-Select (Clontech).

To construct genomic libraries, large segments of genomic DNA are generated by random fragmentation. Examples of appropriate molecular biological techniques and instructions are found in Sambrook et al., Molecular Cloning: A Laboratory Manual, 2nd Ed., Cold Spring Harbor Laboratory, Vols. 1-3 (1989), Methods in Enzymology, Vol. 152: Guide to Molecular Cloning Techniques, Berger and Kimmel, Eds., San Diego: Academic Press, Inc. (1987), Current Protocols in Molecular Biology, Ausubel et al., Eds., Greene Publishing and Wiley-Interscience, New York (1995); Plant Molecular Biology: A Laboratory Manual, Clark, Ed., Springer-Verlag, Berlin (1997). Kits for construction of genomic libraries are also commercially available.

The cDNA or genomic library can be screened using a probe based upon the sequence of a nucleic acid of the present invention such as those disclosed herein. Probes may be used to hybridize with genomic DNA or cDNA sequences to isolate homologous genes in the same or different plant species. Those of skill in the art will appreciate that various degrees of stringency of hybridization can be employed in the assay; and either the hybridization or the wash medium can be stringent. The degree of stringency can be controlled by temperature, ionic strength, pH and the presence of a partially denaturing solvent such as formamide.

Typically, stringent hybridization conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide.

Low stringency conditions include hybridization with a buffer solution of 30% formamide, 1 M NaCl, 1% SDS (sodium dodecyl sulfate) at 37° C., and a wash in 1× to 2× SSC (20× SSC=3.0 M NaCl/0.3 M trisodium citrate) at 50° C. Moderate stringency conditions include hybridization in 40% formamide, 1 M NaCl, 1% SDS at 37° C., and a wash in 0.5× to 1× SSC at 55° C. High stringency conditions include hybridization in 50% formamide, 1 M NaCl, 1% SDS at 37° C., and a wash in 0.1× SSC at 60° C. Hybridization is typically conducted for 4-6 hours.

An extensive guide to the hybridization of nucleic acids is found in Tijssen, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, New York (1993); and Current Protocols in Molecular Biology, Chapter 2, Ausubel et al., Eds., Greene Publishing and Wiley-Interscience, New York (1995).

The nucleic acids of the invention can be amplified from nucleic acid samples using amplification techniques. For instance, polymerase chain reaction (PCR) technology can be used to amplify the sequences of polynucleotides of the present invention and related genes directly from genomic DNA or cDNA libraries. PCR and other in vitro amplification methods may also be useful, for example, to clone nucleic acid sequences that code for proteins to be expressed, to make nucleic acids to use as probes for detecting the presence of the desired mRNA in samples, for nucleic acid sequencing, or for other purposes.

Examples of techniques useful for in vitro amplification methods are found in Berger, Sambrook, and Ausubel, as well as Mullis et al., U.S. Pat. No. 4,683,202 (1987); and, PCR Protocols A Guide to Methods and Applications, Innis et al., Eds., Academic Press Inc., San Diego, Calif. (1990). Commercially available kits for genomic PCR amplification are known in the art. See, e.g., Advantage-GC Genomic PCR Kit (Clontech). The T4 gene 32 protein (Boehringer Mannheim) can be used to improve yield of long PCR products.

PCR-based screening methods have also been described. Wilfinger et al. describe a PCR-based method in which the longest cDNA is identified in the first step so that incomplete clones can be eliminated from study. BioTechniques, 22(3):481-486 (1997).

In one aspect of the invention, nucleic acids can be amplified from a plant library such as a Zea mays nucleic acid library. The nucleic acid library may be a cDNA library, a genomic library, or a library generally constructed from nuclear transcripts at any stage of intron processing.

Libraries can be made from a variety of plant tissues. In maize good results have been obtained using mitotically active tissues such as shoot meristems, shoot meristem cultures, embryos, callus and suspension cultures, immature ears and tassels, and young seedlings. The cDNA of the present invention was obtained from developing endosperm. Since cell cycle proteins are typically expressed at specific cell cycle stages it may be possible to enrich for such rare messages using exemplary cell cycle inhibitors such as aphidicolin, hydroxyurea, mimosine, and double-phosphate starvation methods to block cells at the G1/S boundary. Cells can also be blocked at this stage using the double phosphate starvation method. Hormone treatments that stimulate cell division, for example cytokinin, would also increase expression of cell cycle-related RNAs.

Alternatively, the sequences of the invention can be used to isolate corresponding sequences in other organisms, particularly other plants, more particularly, other monocots. In this manner, methods such as PCR, hybridization, and the like can be used to identify such sequences having substantial sequence similarity to the sequences of the invention. See, for example, Sambrook et al. (1989) Molecular Cloning: A Laboratory Manual (2d ed., Cold Spring Harbor Laboratory Press, Plainview, N.Y.). and Innis et al. (1990), PCR Protocols: A Guide to Methods and Applications (Academic Press, New York). Coding sequences isolated based on their sequence identity to the entire inventive coding sequences set forth herein or to fragments thereof are encompassed by the present invention.

The isolated nucleic acids of the present invention can also be prepared by direct chemical synthesis by methods such as the phosphotriester method of Narang et al., Meth. Enzymol. 68:90-99 (1979); the phosphodiester method of Brown et al., Meth. Enzymol. 68:109-151 (1979); the diethylphosphoramidite method of Beaucage et al., Tetra. Lett. 22:1859-1862 (1981); the solid-phase phosphoramidite triester method described by Beaucage and Caruthers, Tetra. Letts. 22(20):1859-1862 (1981), e.g., using an automated synthesizer, e.g., as described in Needham-VanDevanter et al., Nucleic Acids Res. 12:6159-6168 (1984); and, the solid support method of U.S. Pat. No. 4,458,066. Chemical synthesis generally produces a single stranded oligonucleotide. This may be converted into double stranded DNA by hybridization with a complementary sequence, or by polymerization with a DNA polymerase using the single strand as a template. One of skill will recognize that while chemical synthesis of DNA is limited to sequences of about 100 bases, longer sequences may be obtained by the ligation of shorter sequences.

### EXPRESSION CASSETTES

In another embodiment expression cassettes comprising isolated nucleic acids of the present invention are provided. An expression cassette will typically comprise a polynucleotide of the present invention operably linked to transcriptional initiation regulatory sequences that will direct the transcription of the polynucleotide in the intended host cell, such as tissues of a transformed plant, bacterial or yeast cells.

The construction of expression cassettes that can be employed in conjunction with the present invention is well known to those of skill in the art in light of the present disclosure. See, e.g., Sambrook et al.; Molecular Cloning: A Laboratory Manual; Cold Spring Harbor, N.Y.; (1989); Gelvin et al.; Plant Molecular Biology Manual; (1990); Plant Biotechnology: Commercial Prospects and Problems, eds. Prakash et al.; Oxford & IBH Publishing Co.; New Delhi, India; (1993); and Heslot et al.; Molecular Biology and Genetic Engineering of Yeasts; CRC Press, Inc., USA; (1992); each incorporated herein in its entirety by reference. For example, plant expression vectors may include (1) a cloned plant nucleic acid under the transcriptional control of 5′ and 3′ regulatory sequences and (2) a dominant selectable marker. Such plant expression vectors may also contain, if desired, a promoter regulatory region (e.g., one conferring inducible, constitutive, environmentally- or developmentally-regulated, or cell- or tissue-specific/selective expression), a transcription initiation start site, a ribosome binding site, an RNA processing signal, a transcription termination site, and/or a polyadenylation signal.

Constitutive, tissue-preferred or inducible promoters can be employed. Examples of constitutive promoters include the cauliflower mosaic virus (CaMV) 35S transcription initiation region, the 1′- or 2′-promoter derived from T-DNA of Agrobacterium tumefaciens, the ubiquitin 1 promoter, the Smas promoter, the cinnamyl alcohol dehydrogenase promoter (U.S. Pat. No. 5,683,439), the Nos promoter, the pEmu promoter, the rubisco promoter, the GRP1-8 promoter and other transcription initiation regions from various plant genes known to those of skill.

Examples of inducible promoters are the Adh1 promoter which is inducible by hypoxia or cold stress, the Hsp70 promoter, which is inducible by heat stress, and the PPDK promoter, which is inducible by light. Also useful are promoters that are chemically inducible.

Examples of promoters under developmental control include promoters that initiate transcription preferentially in certain tissues, such as leaves, roots, fruit, seeds, or flowers. An exemplary promoter is the anther specific promoter 5126 (U.S. Pat. Nos. 5,689,049 and 5,689,051). Examples of seed-preferred promoters include, but are not limited to, the 27 kD gamma zein promoter and the waxy promoter, Boronat, A., Martinez, M. C., Reina, M., Puigdomenech, P. and Palau, J.; Isolation and sequencing of a 28 kD glutelin-2 gene from maize: Common elements in the 5′ flanking regions among zein and glutelin genes; Plant Sci. 47:95-102 (1986), and Reina, M., Ponte, I., Guillen, P., Boronat, A. and Palau, J., Sequence analysis of a genomic clone encoding a Zc2 protein from Zea mays W64 A, Nucleic Acids Res. 18:6426 (1990). See the following site relating to the waxy promoter: Kloesgen, R. B., Gierl, A., Schwarz-Sommer, Z S. and Saedler, H., Molecular analysis of the waxy locus of Zea mays, Mol. Gen. Genet 203:237-244 (1986). Promoters that express in the embryo, pericarp, and endosperm are disclosed in WO 00/11177 and WO 00/12733. The disclosures each of these are incorporated herein by reference in their entirety.

Either heterologous or non-heterologous (i.e., endogenous) promoters can be employed to direct expression of the nucleic acids of the present invention. These promoters can also be used, for example, in expression cassettes to drive expression of antisense nucleic acids to reduce, increase, or alter concentration and/or composition of the proteins of the present invention in a desired tissue.

If polypeptide expression is desired, it is generally desirable to include a polyadenylation region at the 3′-end of a polynucleotide coding region. The polyadenylation region can be derived from the natural gene, from a variety of other plant genes, or from T-DNA. The 3′ end sequence to be added can be derived from, for example, the nopaline synthase or octopine synthase genes, or alternatively from another plant gene, or from any other eukaryotic gene.

An intron sequence can be added to the 5′ untranslated region or the coding sequence of the partial coding sequence to increase the amount of the mature message that accumulates. See for example Buchman and Berg, Mol. Cell Biol. 8:4395-4405 (1988); Callis et al., Genes Dev. 1:1183-1200 (1987). Use of maize introns Adh1-S intron 1, 2, and 6, the Bronze-1 intron are known in the art. See generally, The Maize Handbook, Chapter 116, Freeling and Walbot, Eds., Springer, N.Y. (1994).

The vector comprising the sequences from a polynucleotide of the present invention will typically comprise a marker gene which confers a selectable phenotype on plant cells. Usually, the selectable marker gene will encode antibiotic or herbicide resistance. Suitable genes include those coding for resistance to the antibiotic spectinomycin or streptomycin (e.g., the aada gene), the streptomycin phosphotransferase (SPT) gene coding for streptomycin resistance, the neomycin phosphotransferase (NPTII) gene encoding kanamycin or geneticin resistance, the hygromycin phosphotransferase (HPT) gene coding for hygromycin resistance.

Suitable genes coding for resistance to herbicides include those that act to inhibit the action of acetolactate synthase (ALS), in particular the sulfonylurea-type herbicides (e.g., the acetolactate synthase (ALS) gene containing mutations leading to such resistance in particular the S4 and/or Hra mutations), those that act to inhibit action of glutamine synthase, such as phosphinothricin or basta (e.g., the bar gene), or other such genes known in the art. The bar gene encodes resistance to the herbicide basta and the ALS gene encodes resistance to the herbicide chlorsulfuron.

Typical vectors useful for expression of nucleic acids in higher plants are well known in the art and include vectors derived from the tumor-inducing (Ti) plasmid of Agrobacterium tumefaciens described by Rogers et al., Meth. In Enzymol., 153:253-277 (1987). Exemplary A. tumefaciens vectors useful herein are plasmids pKYLX6 and pKYLX7 of Schardl et al., Gene, 61:1-11 (1987) and Berger et al., Proc. Natl. Acad. Sci. USA, 86:8402-8406 (1989). Another useful vector herein is plasmid pBI101.2 that is available from Clontech Laboratories, Inc. (Palo Alto, Calif.).

A variety of plant viruses that can be employed as vectors are known in the art and include cauliflower mosaic virus (CaMV), geminivirus, brome mosaic virus, and tobacco mosaic virus.

A polynucleotide of the present invention can be expressed in either the sense or anti-sense orientation, or in both orientations, as desired. In plant cells, it has been shown that antisense RNA inhibits endogenous gene expression by preventing the accumulation of mRNA encoding the enzyme of interest, see, e.g., Sheehy et al., Proc. Natl. Acad. Sci. USA 85:8805-8809 (1988); and Hiatt et al., U.S. Pat. No. 4,801,340. In plant cells, it has also been shown that inverted repeats, composed of both a sense and antisense sequence, will also effectively silence the targeted gene sequence, see, e.g. Muskens et al., Plant Mol. Biol. 43:243-260 (2000). Another method of suppression is sense suppression. Introduction of nucleic acid configured in the sense orientation has been shown to be an effective means by which to block the transcription of target genes. For an example of the use of this method to modulate expression of endogenous genes see, Napoli et al., Plant Cell 2:279-289 (1990) and U.S. Pat. No. 5,034,323. Recent work has shown suppression with the use of double stranded RNA. Such work is described in Tabara et al., Science 282:5388:430-431 (1998). Hairpin approaches (also referred to as stem loop or inverted repeat sequences) of gene suppression are disclosed in WO 98/53083 and WO 99/53050.

A method of down-regulation of the protein involves using PEST sequences that provide a target for degradation of the protein.

Catalytic RNA molecules or ribozymes can also be used to inhibit expression of plant genes. The inclusion of ribozyme sequences within antisense RNAs confers RNA-cleaving activity upon them, thereby increasing the activity of the constructs. The design and use of target RNA-specific ribozymes is described in Haseloff et al., Nature 334:585-591 (1988).

A variety of cross-linking agents, alkylating agents and radical generating species as pendant groups on polynucleotides of the present invention can be used to bind, label, detect, and/or cleave nucleic acids. For example, Vlassov, V. V., et al., describe covalent bonding of a single-stranded DNA fragment with alkylating derivatives of nucleotides complementary to target sequences Nucleic Acids Res 14:40654076 (1986). A report of similar work by the same group is that by Knorre, D. G., et al., Biochimie 67:785-789 (1985). Iverson and Dervan also showed sequence-specific cleavage of single-stranded DNA mediated by incorporation of a modified nucleotide which was capable of activating cleavage [(J. Am. Chem. Soc. 109:1241-1243 (1987)). Meyer, R. B., et al., J. Am. Chem. Soc. 111:8517-8519 (1989)], affect covalent crosslinking to a target nucleotide using an alkylating agent complementary to the single-stranded target nucleotide sequence. A photoactivated crosslinking to single-stranded oligonucleotides mediated by psoralen was disclosed by Lee, B. L., et al., Biochemistry 27:3197-3203 (1988). Use of crosslinking in triple-helix forming probes was also disclosed by Home, et al., J. Am. Chem. Soc. 112:2435-2437 (1990). Use of N4, N4-ethanocytosine as an alkylating agent to crosslink to single-stranded oligonucleotides has also been described by Webb and Matteucci, J. Am. Chem. Soc. 108:2764-2765 (1986); Nucleic Acids Res 14:7661-7674 (1986); Feteritz et al., J. Am. Chem. Soc. 113:4000 (1991). Various compounds to bind, detect, label, and/or cleave nucleic acids are known in the art. See, for example, U.S. Pat. Nos. 5,543,507; 5,672,593; 5,484,908; 5,256,648; and, 5,681,941.

### PROTEINS

Proteins of the present invention include proteins derived from the native protein by deletion (so-called truncation), and addition or substitution of one or more amino acids at one or more sites in the native protein. Such variants may result from, for example, genetic polymorphism or from human manipulation. Methods for such manipulations are generally known in the art.

For example, amino acid sequence variants of the polypeptide can be prepared by mutations in the cloned DNA sequence encoding the native protein of interest. Methods for mutagenesis and nucleotide sequence alterations are well known in the art. See, for example, Walker and Gaastra, eds. Techniques in Molecular Biology (MacMillan Publishing Company, New York) (1983); Kunkel, Proc. Natl. Acad. Sci. USA 82:488-492 (1985); Kunkel et al., Methods Enzymol. 154:367-382 (1987); Sambrook et al., Molecular Cloning: A Laboratory Manual (Cold Spring Harbor, N.Y.) (1989); U.S. Pat. No. 4,873,192; and the references cited therein; herein incorporated by reference. Guidance as to appropriate amino acid substitutions that do not affect biological activity of the protein of interest may be found in the model of Dayhoff et al., Atlas of Protein Sequence and Structure (Natl. Biomed. Res. Found., Washington, D.C.) (1978), herein incorporated by reference. Conservative substitutions, such as exchanging one amino acid with another having similar properties, may be preferred.

In constructing variants of the proteins of interest, modifications to the nucleotide sequences encoding the variants will be made such that variants continue to possess the desired activity. When protein expression is desired, any mutations made in the DNA encoding the variant protein must not place the sequence out of reading frame and preferably will not create complementary regions that could produce secondary mRNA structure. See EP Patent Application Publication No. 75,444.

The isolated proteins of the present invention include a polypeptide comprising at least 25 contiguous amino acids encoded by any one of the nucleic acids of the present invention, or polypeptides which are conservatively modified variants thereof. The proteins of the present invention or variants thereof can comprise any number of contiguous amino acid residues from a polypeptide of the present invention, wherein that number is selected from the group of integers consisting of from 25 to the number of residues in a full-length polypeptide of the present invention. Optionally, this subsequence of contiguous amino acids is at least 30, 35, 40, 50, 60, 70, 80, 90, 100, 150, 200, or 300 amino acids in length.

The present invention includes catalytically active polypeptides (i.e., enzymes). Catalytically active polypeptides will generally have a specific activity of at least 20%, 30%, 40%, 50%, 60%, 70%, 80%, 90%, or 95% that of the native (non-synthetic), endogenous polypeptide. Further, the substrate specificity (k_{cat}/Kₘ) is optionally substantially similar to the native (non-synthetic), endogenous polypeptide. Typically, the Kₘ will be at least 30%, 40%, 50%, 60%, 70%, 80%, 90% or 95%. Methods of assaying and quantifying measures of enzymatic activity and substrate specificity (k_{cat}/Kₘ), are well known to those of skill in the art.

The present invention includes modifications that can be made to an inventive protein without diminishing its biological/catalytic activity. Some modifications may be made to facilitate the cloning, expression, or incorporation of the targeting molecule into a fusion protein. Such modifications are well known to those of skill in the art and include, for example, a methionine added at the amino terminus to provide an initiation site, or additional amino acids (e.g., poly His) placed on either terminus to create conveniently located restriction sites or termination codons or purification sequences.

A protein of the present invention can be expressed in a recombinantly engineered cell such as bacterial, yeast, insect, mammalian, or plant cells. The cells produce the protein in a non-natural condition (e.g., in quantity, composition, location, and/or time), because they have been genetically altered through human intervention to do so.

Typically, an intermediate host cell will be used in the practice of this invention to increase the copy number of the cloning vector. With an increased copy number, the vector containing the nucleic acid of interest can be isolated in significant quantities for introduction into the desired plant cells.

Host cells that can be used in the practice of this invention include prokaryotes, including bacterial hosts such as Eschericia coli, Salmonella typhimurium, and Serratia marcescens. Eukaryotic hosts such as yeast or filamentous fungi may also be used in this invention. It may be convenient to use plant promoters that do not cause expression of the polypeptide in bacteria.

Commonly used prokaryotic control sequences include promoters such as the beta lactamase (penicillinase) and lactose (lac) promoter systems [Chang et al., Nature 198:1056 (1977)], the tryptophan (trp) promoter system [Goeddel et al., Nucleic Acids Res. 8:4057 (1980)] and the lambda derived P L promoter and N-gene ribosome binding site [Shimatake et al., Nature 292:128 (1981)]. The inclusion of selection markers in DNA vectors transfected in E. coli is also useful. Examples of such markers include genes specifying resistance to ampicillin, tetracycline, or chloramphenicol.

The vector is selected to allow introduction into the appropriate host cell. Bacterial vectors are typically of plasmid or phage origin. Expression systems for expressing a protein of the present invention are available using Bacillus sp. and Salmonella [Palva et al., Gene 22:229-235 (1983); Mosbach et al., Nature 302:543-545 (1983)].

Synthesis of heterologous proteins in yeast is well known. See Sherman, F., et al., Methods in Yeast Genetics, Cold Spring Harbor Laboratory (1982). Two widely utilized yeast for production of eukaryotic proteins are Saccharomyces cerevisiae and Pichia pastoris. Vectors, strains, and protocols for expression in Saccharomyces and Pichia are known in the art and available from commercial suppliers (e.g., Invitrogen). Suitable vectors usually have expression control sequences, such as promoters, including 3-phosphoglycerate kinase or alcohol oxidase, and an origin of replication, termination sequences and the like as desired.

A protein of the present invention, once expressed, can be isolated from yeast by lysing the cells and applying standard protein isolation techniques to the lysates. The monitoring of the purification process can be accomplished by using western blot techniques or radioimmunoassay of other standard immunoassay techniques.

The proteins of the present invention can also be constructed using non-cellular synthetic methods. Solid-phase synthesis of proteins of less than about 50 amino acids in length may be accomplished by attaching the C-terminal amino acid of the sequence to an insoluble support followed by sequential addition of the remaining amino acids in the sequence. Techniques for solid-phase synthesis are described by Barany and Merrifield, Solid-Phase Peptide Synthesis, pp. 3-284 in The Peptides: Analysis, Synthesis, Biology, Vol. 2: Special Methods in Peptide Synthesis, Part A; Merrifield, et al., J. Am. Chem. Soc. 85: 2149-2156 (1963), and Stewart et al., Solid Phase Peptide Synthesis, 2nd ed., Pierce Chem. Co., Rockford, Ill. (1984). Proteins of greater length may be synthesized by condensation of the amino and carboxy termini of shorter fragments. Methods of forming peptide bonds by activation of a carboxy terminal end (e.g., by the use of the coupling reagent N,N′-dicycylohexylcarbodiimide) is known to those of skill.

The proteins of this invention may be obtained in substantial purity by standard techniques well known in the art, including detergent solubilization, selective precipitation with such substances as ammonium sulfate, column chromatography, immunopurification methods, and others. See, for instance, R. Scopes, Protein Purification: Principles and Practice, Springer-Verlag: New York (1982); Deutscher, Guide to Protein Purification, Academic Press (1990). For example, antibodies may be raised to the proteins as described herein. Purification from E. coli can be achieved following procedures described in U.S. Pat. No. 4,511,503. Detection of the expressed protein is achieved by methods known in the art and include, for example, radioimmunoassays, western blotting techniques or immunoprecipitation.

The present invention further provides a method for modulating (i.e., increasing or decreasing) the concentration or composition of the polypeptides of the present invention in a plant or part thereof. Modulation of the polypeptides can be effected by increasing or decreasing the concentration and/or the composition of the polypeptides in a plant. The method comprises transforming a plant cell with an expression cassette comprising a polynucleotide of the present invention to obtain a transformed plant cell, growing the transformed plant cell under plant forming conditions, and inducing expression of the polynucleotide in the plant for a time sufficient to modulate concentration and/or composition of the polypeptides in the plant or plant part.

In one embodiment of the present invention, cell cycle gene expression could increase or decrease cell growth. It could also increase or decrease cell division, alter the percentage of time that cells of the plant are arrested or change the amount of time in a particular portion of the cell cycle. Expression of cell cycle genes can increase growth and growth rate, increase plant height and or size, also increase crop yield, and the number of pods per plant. It can also provide a positive growth advantage and can enhance or inhibit organ growth (e.g. seed, root, shoot, ear, tassel, stalk, pollen, stamen (male sterility), parthenocarpic fruits, organ oblation). Cell cycle gene expression can increase transformation efficiency, enhance embryogenic response (size and growth rate), increase induction of callus, provide a positive selection method, use of co-transformation, and increase plant regeneration. Expression of cell cycle genes can increase the number of pods/plant, increase seeds/pod, and alter lag time in seed development. It can improve response to environmental stress (e.g. drought, heat, cold) and provide hormone independent growth. Expression of cell cycle genes can be altered and all or portions of genes removed using FLP/FRT, PEST, altered PEST sequence, Rb binding site, or the cyclin box. Patterns of expression can also be altered by using a variety of promoters including inducible promoters (e.g. inducible chemically, hormonally in, tissue specific). Expression of cell cycle genes can be in planta or in vitro. For example, cell cycle gene expression in bioreactors can increase growth rate and production of protein or other products.

In some embodiments, the content and/or composition of polypeptides of the present invention in a plant may be modulated by altering, in vivo or in vitro, the promoter of a non-isolated gene of the present invention to up- or down-regulate gene expression. In some embodiments, the coding regions of native genes of the present invention can be altered via substitution, addition, insertion, or deletion to decrease activity of the encoded enzyme. See, e.g., Kmiec, U.S. Pat. 5,565,350; Zarling et al., PCT/US93/03868.

In particular, modulating cell cycle proteins is expected to provide a positive growth advantage and increase crop yield. Cell cycle nucleic acids can be adducted to a second nucleic acid sequence encoding a DNA-binding domain, for use in two-hybrid systems to identify RB3-interacting proteins. It is expected that modulating the level of cell cycle protein, i.e. reducing RB3 expression will increase growth of specific tissues in which this occurs, for example in the ear or more specifically in the seed. Also, modulating cell cycle proteins affects the cell number in a tissue of a plant, thereby affecting the size and characteristics of that tissue organ. Modulation could affect any plant tissue such as, but not limited to root, seed, tassel, ear, silk, stalk, embryo, flower, grain, germ, head, leave, stem, seed, trunk, meristem or fruit. Changes in plant tissue will influence quality traits, agronomic traits and susceptibility to disease and insects.

In some embodiments, an isolated nucleic acid (e.g., a vector) comprising a promoter sequence is transfected into a plant cell. Subsequently, a plant cell comprising the isolated nucleic acid is selected for by means known to those of skill in the art such as, but not limited to, Southern blot, DNA sequencing, or PCR analysis using primers specific to the promoter and to the nucleic acid and detecting amplicons produced therefrom. A plant or plant part altered or modified by the foregoing embodiments is grown under plant forming conditions for a time sufficient to modulate the concentration and/or composition of polypeptides of the present invention in the plant. Plant forming conditions are well known in the art.

In general, concentration of the polypeptides is increased or decreased by at least 5%, 10%, 20%, 30%, 40%, 50%, 60%, 70%, 80%, or 90% relative to a native control plant, plant part, or cell lacking the aforementioned expression cassette. Modulation in the present invention may occur during and/or subsequent to growth of the plant to the desired stage of development.

Modulating nucleic acid expression temporally and/or in particular tissues can be controlled by employing the appropriate promoter operably linked to a polynucleotide of the present invention in, for example, sense or antisense orientation as discussed in greater detail above. Induction of expression of a polynucleotide of the present invention can also be controlled by exogenous administration of an effective amount of inducing compound. Inducible promoters and inducing compounds that activate expression from these promoters are well known in the art.

The polypeptides of the present invention can be modulated in monocots, preferably cereals, or dicots. Typical plants include corn, soybean, sunflower, sorghum, canola, wheat, alfalfa, cotton, rice, barley, oil-seed Brassica or millet. In another embodiment the polypeptides of this present invention are modulated in bacteria and yeast.

Means of detecting the proteins of the present invention are not critical aspects of the present invention. The proteins can be detected and/or quantified using any of a number of well recognized immunological binding assays (see, e.g., U.S. Pat. Nos. 4,366,241; 4,376,110; 4,517,288; and 4,837,168). For a review of the general immunoassays, see also Methods in Cell Biology, Vol. 37: Antibodies in Cell Biology, Asai, Ed., Academic Press, Inc. New York (1993); Basic and Clinical Immunology 7th Edition, Stites & Terr, Eds. (1991). Moreover, the immunoassays of the present invention can be performed in any of several configurations, e.g., those reviewed in Enzyme Immunoassay, Maggio, Ed., CRC Press, Boca Raton, Fla. (1980); Tijan, Practice and Theory of Enzyme Immunoassays, Laboratory Techniques in Biochemistry and Molecular Biology, Elsevier Science Publishers B.V., Amsterdam (1985); Harlow and Lane, supra; Immunoassay: A Practical Guide, Chan, Ed., Academic Press, Orlando, Fla. (1987); Principles and Practice of Immunoassays, Price and Newman Eds., Stockton Press, NY (1991); and Non-isotopic Immunoassays, Ngo, Ed., Plenum Press, NY (1988).

Typical methods for detecting proteins include western blot (immunoblot) analysis, analytic biochemical methods such as electrophoresis, capillary electrophoresis, high-performance liquid chromatography (HPLC), thin-layer chromatography (TLC), hyperdiffusion chromatography, and the like, and various immunological methods such as fluid or gel precipitin reactions, immunodiffusion (single or double), immunoelectrophoresis, radioimmunoassays (RIAs), enzyme-linked immunosorbent assays (ELISAs), immunofluorescent assays, and the like.

Non-radioactive labels are often attached by indirect means. Generally, a ligand molecule (e.g., biotin) is covalently bound to the molecule. The ligand then binds to an anti-ligand (e.g., streptavidin) molecule, which is either inherently detectable or covalently bound to a signal system, such as a detectable enzyme, a fluorescent compound, or a chemiluminescent compound. A number of ligands and anti-ligands can be used. Where a ligand has a natural anti-ligand, for example, biotin, thyroxine, and cortisol, it can be used in conjunction with the labeled, naturally occurring anti-ligands. Alternatively, any haptenic or antigenic compound can be used in combination with an antibody.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidoreductases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal-producing systems that may be used, see, U.S. Pat. No. 4,391,904, which is incorporated herein by reference.

Some assay formats do not require the use of labeled components. For instance, agglutination assays can be used to detect the presence of the target antibodies. In this case, antigen-coated particles are agglutinated by samples comprising the target antibodies. In this format, none of the components need be labeled and the presence of the target antibody is detected by simple visual inspection.

The proteins of the present invention can be used for identifying compounds that bind to (e.g., substrates), and/or increase or decrease (i.e., modulate) the enzymatic activity of, catalytically active polypeptides of the present invention. The method comprises contacting a polypeptide of the present invention with a compound whose ability to bind to or modulate enzyme activity is to be determined. The polypeptide employed will have at least 20%, 30%, 40%, 50%, 60%, 70%, 80%, or 90% of the specific activity of the native, full-length polypeptide of the present invention (e.g., enzyme). Methods of measuring enzyme kinetics are well known in the art. See, e.g., Segel, Biochemical Calculations, 2^{nd} ed., John Wiley and Sons, New York (1976).

Antibodies can be raised to a protein of the present invention, including individual, allelic, strain, or species variants, and fragments thereof, both in their naturally occurring (full-length) forms and in recombinant forms. Additionally, antibodies are raised to these proteins in either their native configurations or in non-native configurations. Anti-idiotypic antibodies can also be generated. Many methods of making antibodies are known to persons of skill.

In some instances, it is desirable to prepare monoclonal antibodies from various mammalian hosts, such as mice, rodents, primates, humans, etc. Description of techniques for preparing such monoclonal antibodies are found in, e.g., Basic and Clinical Immunology, 4th ed., Stites et al., Eds., Lange Medical Publications, Los Altos, Calif., and references cited therein; Harlow and Lane, Supra; Goding, Monoclonal Antibodies: Principles and Practice, 2nd ed., Academic Press, New York, N.Y. (1986); and Kohler and Milstein, Nature 256:495-497 (1975).

Other suitable techniques involve selection of libraries of recombinant antibodies in phage or similar vectors [see, e.g., Huse et al., Science 246:1275-1281 (1989); and Ward et al., Nature 341:544-546 (1989); and Vaughan et al., Nature Biotechnology 14:309-314 (1996)]. Alternatively, high-avidity human monoclonal antibodies can be obtained from transgenic mice comprising fragments of the unrearranged human heavy and light chain Ig loci (i.e., minilocus transgenic mice) [Fishwild et al., Nature Biotech. 14:845-851 (1996)]. Also, recombinant immunoglobulins may be produced. See, Cabilly, U.S. Pat. No. 4,816,567; and Queen et al., Proc. Natl. Acad. Sci. 86:10029-10033 (1989).

The antibodies of this invention can be used for affinity chromatography in isolating proteins of the present invention, for screening expression libraries for particular expression products such as normal or abnormal protein or for raising anti-idiotypic antibodies which are useful for detecting or diagnosing various pathological conditions related to the presence of the respective antigens.

Frequently, the proteins and antibodies of the present invention will be labeled by joining, either covalently or non-covalently, a substance that provides for a detectable signal. A wide variety of labels and conjugation techniques are known and are reported extensively in both the scientific and patent literatures. Suitable labels include radionucleotides, enzymes, substrates, cofactors, inhibitors, fluorescent moieties, chemiluminescent moieties, magnetic particles, and the like.

Transfection/Transformation of Cells

The method of transformation/transfection is not critical to the invention; various methods of transformation or transfection are currently available. As newer methods are available to transform crops or other host cells they may be directly applied. Accordingly, a wide variety of methods have been developed to insert a DNA sequence into the genome of a host cell to obtain the transcription and/or translation of the sequence to effect phenotypic changes in the organism. Thus, any method that provides for efficient transformation/transfection may be employed.

A DNA sequence coding for the desired polynucleotide of the present invention, for example a cDNA, RNA or a genomic sequence, will be used to construct an expression cassette that can be introduced into the desired plant. Isolated nucleic acid acids of the present invention can be introduced into plants according to techniques known in the art. Generally, expression cassettes as described above and suitable for transformation of plant cells are prepared.

Techniques for transforming a wide variety of higher plant species are well known and described in the technical, scientific, and patent literature. See, for example, Weisinger et al., Ann. Rev. Genet. 22:421-477 (1988). For example, the DNA construct may be introduced directly into the genomic DNA of the plant cell using techniques such as electroporation, PEG-mediated transfection, particle bombardment, silicon-fiber delivery, or microinjection of plant cell protoplasts or embryogenic callus. See, e.g., Tomes et al., Direct DNA Transfer into Intact Plant Cells Via Microprojectile Bombardment. pp. 197-213 in Plant Cells Tissue and Organ Culture, Fundamental Methods. eds. O. L. Gamborg and G. C. Phillips. Springer-Verlag Berlin Heidelberg N.Y., 1995.

The introduction of DNA constructs using polyethylene glycol precipitation is described in Paszkowski et al., Embo J. 3:2717-2722 (1984). Electroporation techniques are described in Fromm et al., Proc. Natl. Acad. Sci USA 82:5824 (1985). Ballistic transformation techniques are described in Klein et al., Nature 327:70-73 (1987).

Agrobacterium tumefaciens-meditated transformation techniques are well described in the scientific literature. See, for example Horsch et al., Science 233:496-498 (1984), and Fraley et al., Proc. Natl. Acad. Sci. USA 80:4803 (1983). For instance, Agrobacterium transformation of maize is described in U.S. Pat. No. 5,550,318 and U.S. Pat. No. 5,981,840. The DNA constructs may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria. See, U.S. Pat. No. 5,591,616. Also, a combination of particle bombardment and Agrobacterium host is found in U.S. Pat. No. 5,932,782.

Other methods of transfection or transformation include (1) Agrobacterium rhizogenes-mediated transformation (see, e.g., Lichtenstein and Fuller In: Genetic Engineering, vol. 6, PWJ Rigby, Ed., London, Academic Press, 1987; and Lichtenstein, C. P., and Draper, J,. In: DNA Cloning, Vol. II, D. M. Glover, Ed., Oxford, IRI Press, 1985) [Application PCT/US87/02512 (WO 88/02405 published Apr. 7, 1988) describes the use of A. rhizogenes strain A4 and its Ri plasmid along with A. tumefaciens vectors pARC8 or pARC16 (2) liposome-mediated DNA uptake (see, e.g., Freeman et al., Plant Cell Physiol. 25:1353,1984), (3) the vortexing method (see, e.g., Kindle, Proc. Natl. Acad. Sci. USA 87:1228, (1990).

DNA can also be introduced into plants by direct DNA transfer into pollen as described by Zhou et al., Methods in Enzymology 101:433 (1983); D. Hess, Intern. Rev. Cytol., 107:367 (1987); Luo et al., Plant Mol. Biol. Reporter 6:165 (1988). Expression of polypeptide coding nucleic acids can be obtained by injection of the DNA into reproductive organs of a plant as described by Pena et al., Nature 325:274 (1987). DNA can also be injected directly into the cells of immature embryos and the rehydration of desiccated embryos as described by Neuhaus et al., Theor. Appl. Genet. 75:30 (1987); and Benbrook et al., in Proceedings Bio Expo 1986, Butterworth, Stoneham, Mass., pp. 27-54 (1986).

Animal and lower eukaryotic (e.g., yeast) host cells are competent or rendered competent for transfection by various means. There are several well-known methods of introducing DNA into animal cells. These include: calcium phosphate precipitation, fusion of the recipient cells with bacterial protoplasts containing the DNA, treatment of the recipient cells with liposomes containing the DNA, DEAE dextran, electroporation, biolistics, and micro-injection of the DNA directly into the cells. The transfected cells are cultured by means well known in the art. Kuchler, R. J., Biochemical Methods in Cell Culture and Virology, Dowden, Hutchinson and Ross, Inc. (1977).

Transgenic Plant Regeneration

Transformed plant cells which are derived by any of the above transformation techniques can be cultured to regenerate a whole plant that possesses the transformed genotype. Such regeneration techniques often rely on manipulation of certain phytohormones in a tissue culture growth medium, typically relying on a biocide and/or herbicide marker which has been introduced together with a polynucleotide of the present invention. For regeneration of maize see McCormick et al., Plant Cell Reports 5:86-89 (1986).

Plants cells transformed with a plant expression vector can be regenerated, e.g., from single cells, callus tissue or leaf discs according to standard plant tissue culture techniques. It is well known in the art that various cells, tissues, and organs from almost any plant can be successfully cultured to regenerate an entire plant. Plant regeneration from cultured protoplasts is described in Evans et al., Protoplasts Isolation and Culture, Handbook of Plant Cell Culture, Macmillan Publishing Company, New York, pp. 124-176 (1983); and Binding, Regeneration of Plants, Plant Protoplasts, CRC Press, Boca Raton, pp. 21-73 (1985).

The regeneration of plants containing the foreign gene introduced by Agrobacterium can be achieved as described by Horsch et al., Science, 227:1229-1231 (1985) and Fraley et al., Proc. Natl. Acad. Sci. USA 80:4803 (1983). This procedure typically produces shoots within two to four weeks and these transformant shoots are then transferred to an appropriate root-inducing medium containing the selective agent and an antibiotic to prevent bacterial growth. Transgenic plants of the present invention may be fertile or sterile.

Regeneration can also be obtained from plant callus, explants, organs, or parts thereof. Such regeneration techniques are described generally in Klee et al., Ann. Rev. of Plant Phys. 38:467-486 (1987). The regeneration of plants from either single plant protoplasts or various explants is well known in the art. See, for example, Methods for Plant Molecular Biology, A. Weissbach and H. Weissbach, eds., Academic Press, Inc., San Diego, Calif. (1988). For maize cell culture and regeneration see, generally, The Maize Handbook, Freeling and Walbot, Eds., Springer, New York (1994); Corn and Corn Improvement, 3^{rd} edition, Sprague and Dudley Eds., American Society of Agronomy, Madison, Wis. (1988).

One of skill will recognize that after the expression cassette is stably incorporated in transgenic plants and confirmed to be operable, it can be introduced into other plants by sexual crossing. Any of a number of standard breeding techniques can be used, depending upon the species to be crossed.

In vegetatively-propagated crops, mature transgenic plants can be propagated by the taking of cuttings or by tissue culture techniques to produce multiple identical plants. Selection of desirable transgenics is made and new varieties are obtained and propagated vegetatively for commercial use. In seed-propagated crops, mature transgenic plants can be self-crossed to produce a homozygous inbred plant. The inbred plant produces seed containing the newly introduced heterologous nucleic acid. These seeds can be grown to produce plants that would produce the selected phenotype.

Parts obtained from the regenerated plant, such as flowers, seeds, leaves, branches, fruit, and the like are included in the invention, provided that these parts comprise cells containing the isolated nucleic acid of the present invention. Progeny and variants, and mutants of the regenerated plants are also included within the scope of the invention, provided that these parts comprise the introduced nucleic acid sequences.

Transgenic plants expressing a selectable marker can be screened for transmission of the nucleic acid of the present invention by, for example, standard immunoblot and DNA detection techniques. Transgenic lines are also typically evaluated on levels of expression of the heterologous nucleic acid. Expression at the RNA level can be determined initially to identify and quantitate expression-positive plants. Standard techniques for RNA analysis can be employed and include PCR amplification assays using oligonucleotide primers designed to amplify only the heterologous RNA templates and solution hybridization assays using heterologous nucleic acid-specific probes. The RNA-positive plants can then be analyzed for protein expression by western immunoblot analysis using the specifically reactive antibodies of the present invention. In addition, in situ hybridization and immunocytochemistry according to standard protocols can be done using heterologous nucleic acid-specific polynucleotide probes and antibodies, respectively, to localize sites of expression within transgenic tissue. Generally, a number of transgenic lines are usually screened for the incorporated nucleic acid to identify and select plants with the most appropriate expression profiles.

In one embodiment a transgenic plant is provided which is homozygous for the added heterologous nucleic acid; i.e., a transgenic plant that contains two added nucleic acid sequences, one gene at the same locus on each chromosome of a chromosome pair. A homozygous transgenic plant can be obtained by sexually mating (selfing) a heterozygous transgenic plant that contains a single added heterologous nucleic acid, germinating some of the seed produced and analyzing the resulting plants produced for altered expression of a polynucleotide of the present invention relative to a control plant (i.e., native, nontransgenic). Back-crossing to a parental plant and out-crossing with a non-transgenic plant are also contemplated.

The present invention provides a method of genotyping a plant comprising a polynucleotide of the present invention. Genotyping provides a means of distinguishing homologs of a chromosome pair and can be used to differentiate segregants in a plant population. Molecular marker methods can be used for phylogenetic studies, characterizing genetic relationships among crop varieties, identifying crosses or somatic hybrids, localizing chromosomal segments affecting monogenic traits, map based cloning, and the study of quantitative inheritance. See, e.g., Plant Molecular Biology: A Laboratory Manual, Chapter 7, Clark, Ed., Springer-Verlag, Berlin (1997). For molecular marker methods, see generally, The DNA Revolution by Andrew H. Paterson (Chapter 2) in: Genome Mapping in Plants (ed. Andrew H. Paterson) by Academic Press/R. G. Landis Company, Austin, Tex., pp. 7-21 (1996).

The particular method of genotyping in the present invention may employ any number of molecular marker analytic techniques such as, but not limited to, restriction fragment length polymorphisms (RFLPs). RFLPs are the product of allelic differences between DNA restriction fragments caused by nucleotide sequence variability. Thus, the present invention further provides a means to follow segregation of a gene or nucleic acid of the present invention as well as chromosomal sequences genetically linked to these genes or nucleic acids using such techniques as RFLP analysis.

Plants that can be used in the methods of the invention include monocotyledonous and dicotyledonous plants. Typical plants include corn, soybean, sunflower, sorghum, canola, wheat, alfalfa, cotton, rice, barley, oil-seed Brassica and millet.

Seeds derived from plants regenerated from transformed plant cells, plant parts or plant tissues, or progeny derived from the regenerated transformed plants, may be used directly as feed or food, or further processing may occur.

Expression of the inventive nucleic acids in plants, such as corn is expected to enhance growth and biomass accumulation. Other more specialized applications exist for these nucleic acids at the whole plant level. It has been demonstrated that endoreduplication occurs in numerous cell types within plants. Under the direction of selected promoters, expression of cell cycle genes (and possibly expression of such genes in conjunction with genes that inhibit mitosis) will further stimulate the process of endoreduplication.

Rb3 can be used in a two-hybrid system to identify maize genes involved in control of cell division. Rb3 gene expression plays a prominent role as an anti-proliferative agent, inhibiting cell cycle progression. The eukaryotic cell cycle is controlled by the sequential activation and inhibition of a phosphorylation cascade centered around a suite of regulatory kinases called cyclin dependant kinases, and the respective stage-specific cyclins that associate with the CDKs. The onset of mitosis, and the onset and maintenance of S-phase are marked by a high level of CDK activity.

The protein encoded by the Rb3 gene is integrally involved in regulating cell division, and likely plays a role in differentiation and apoptosis pathways in specific cell types. These cellular decisions are mediated by RB3 generally acting with other proteins to repress transcription, for example, through association with E2F, the recruitment of histone deacetylase or heterochomatin-associated proteins that mediate chromatin condensation. As such, the Rb3 gene and its encoded protein can potentially be used to identify other proteins involved in the above processes. This can be done using the Rb3 gene as bait (the target fused to the DNA-binding domain) in a yeast two-hybrid screen. Methods for two-hybrid library construction, cloning of the reporter gene, cloning of the DNA-binding and activation domain hybrid gene cassettes, yeast culture, and transformation of the yeast are all done according to well-established methods (see Sambrook et al., 1990; Ausubel et al., 1990; Hannon and Bartels, 1995). When maize Rb3 is used as bait in such a two-hybrid screen, proteins that interact with RB3 such as E2F, histone deacetylase and various chromatin-associated proteins are identified.

Regardless of the method of DNA delivery, cells competent for the integration of foreign DNA are typically actively dividing. There is a growing body of evidence suggesting that integration of foreign DNA occurs in dividing cells (this includes both Agrobacterium and direct DNA delivery methods). It has long been observed that dividing transformed cells represent only a fraction of cells that transiently express a transgene. It is well known (in non-plant systems) that the delivery of damaged DNA, (similar to what we introduce by particle gun delivery methods) induces checkpoint controls and inhibits cell cycle progression. Cell cycle blockage is typically regulated by proteins such as RB3. This inhibition can be obviated by transient down-regulation of negative regulators such as RB3. Regardless of the mechanism of arrest; i.e. presence of damaged DNA or delivery into a non-cycling differentiated cell, stimulation of the cell cycle will increase integration frequencies.

Transient expression of the Rb3-antisense down-regulates RB3 which in turn releases the cells to progress through the cell cycle and divide. This effectively overcomes the G1/S checkpoint controls, and increases the proportion of recipient cells (i.e. into which DNA was introduced) that enter S-phase. This stimulation through the G1/S transition in cells harboring transgenic plasmid DNA, or the corresponding polypeptide, provides an optimal cellular environment for integration of the introduced genes.

An alternative to conventional antisense strategies is the use of antisense oligonucleotides (often with chemically-modified nucleotides). Such an antisense oligonucleotide, typically a 15-18 mer (but this size can vary either more or less), is designed to bind around accessible regions such as the ribosomal binding site around the “Start” codon. Introduction of the antisense oligonucleotide into a cell will transiently stop expression of the targeted gene.

In cells that receive such an antisense oligonucleotide targeted to Rb3, the antisense oligonucleotide transiently disrupts RB3 expression and stimulates entry into S-phase [(as observed in mammalian cells—see Nuell et al. Mol. Cell. Biol. 11:1372-1381 (1991).].

Antibodies directed against RB3 can also be used to mitigate RB3's cell cycle-inhibitory activity, thus stimulating the cell cycle and transgene integration. Genes encoding single chain antibodies, expressed behind a suitable promoter, for example the ubiquitin promoter, could be used in such a fashion. Transient expression of an anti-RB3 antibody could temporarily disrupt normal RB3 function (i.e. preventing its repressive binding to E2F) and thus stimulate the cell cycle. Alternatively, antibodies raised against RB3 could be purified and used for direct introduction into maize cells.

The methods above represent various means of using the Rb3-antisense or anti-RB3 antibodies, or antisense oligonucleotides to transiently stimulate DNA replication and cell division, which in turn enhances transgene integration by providing an improved cellular/molecular environment for this event to occur.

Methods are contemplated wherein the Rb3 nucleic acids, fragments, and/or polypeptides are used to improve transformation.

Based on results in other eukaryotes, expression of the Rb3 gene should block the G1/S transition and prevent cell division. This decrease in division rate is assessed in a number of different manners, being reflected in larger cell size, less rapid incorporation of radiolabeled nucleotides, and slower growth (i.e. less biomass accumulation). Conversely, expression of Rb3 antisense (or an appropriate antisense oligonucleotide, or anti-RB3 antibody) will result in smaller cells, more rapid incorporation of radiolabeled nucleotides, and faster growth.

Rb3-antisense expression using tissue-specific or cell-specific promoters stimulates cell cycle progression in the expressing tissues or cells. For example, using a seed-specific promoter will stimulate cell division rate and result in increased seed biomass. Alternatively, driving Rb3-antisense expression with a strongly-expressed, early tassel-specific promoter will enhance development of this entire reproductive structure. Expression of Rb3 antisense in other cell types and/or at different stages of development will similarly stimulate cell division rates.

Meristem transformation protocols rely on the transformation of apical initials or cells that can become apical initials following reorganization due to injury or selective pressure. The progenitors of these apical initials differentiate to form the tissues and organs of the mature plant (i.e. leaves, stems, ears, tassels, etc.). The meristems of most angiosperms are layered, with each layer having its own set of initials. Normally in the shoot apex these layers rarely mix. In maize the outer layer of the apical meristem, the L1, differentiates to form the epidermis while descendents of cells in the inner layer, the L2, give rise to internal plant parts, including the gametes. The initials in each of these layers are defined solely by position and can be replaced by adjacent cells if they are killed or compromised. Meristem transformation frequently targets a subset of the population of apical initials and the resulting plants are chimeric. If for example, 1 of 4 initials in the L1 layer of the meristem are transformed, only ¼ of the epidermis would be transformed. Selective pressure can be used to enlarge sectors but this selection must be non-lethal, since large groups of cells are required for meristem function and survival.

Transformation of an apical initial with a Rb3-antisense sequence under the expression of a promoter active in the apical meristem (either meristem-specific or constitutive) would allow the transformed cells to grow faster and displace wild-type initials driving the meristem towards homogeneity and minimizing the chimeric nature of the plant body.

Transient expression of the Rb3-antisense sequence in meristem cells, through stimulation of the G1/S transition, will result in greater integration frequencies and hence more numerous transgenic sectors. Integration and expression of the Rb3-antisense sequence will impart a competitive advantage to expressing cells resulting in a progressive enlargement of the transgenic sector. Due to the enhanced growth rate in Rb3-antisense-expressing meristem cells, they will supplant wild-type meristem cells as the plant continues to grow. The result will be both enlargement of transgenic sectors within a given cell layer (i.e. periclinal expansion) and into adjacent cell layers (i.e. anticlinal invasions). As cells expressing the Rb3-antisense occupy an increasingly large proportion of the meristem, the frequency of transgene germline inheritance increases accordingly.

Tissue- and temporal-specific gene expression and regulation is found, inter alia, during sexual reproduction in eukaryotes. In plant gametogenesis, important cytological and biochemical changes occur during pollen development when the asymmetric mitotic division of the haploid microspore results in the formation of two cells each with different developmental fates. The vegetative cell supports pollen growth while the generative cell undergoes mitosis and develops into sperm cells. Messenger RNAs specific to both pathways within pollen have been identified in plants such as maize, tomato, tobacco, rice and pansy; and mRNAs specific to pollen or to one or more other cell types within anther such as tapetum, epidermis and stomium have also been identified.

It is envisioned that the use of tissue-specific promoters, such as MS45 (WO 98/59061) could limit inducibility to specific tissues or cell types. Additionally, the EcR/USP system (WO 00/15791) with tissue-specific promoters could limit expression to given sites within an organism and facilitate the targeting of gene expression to just those given sites or developmental stages. The system could thus be used to selectively induce expression in only the seed or in only the reproductive structures of a plant without expression in other areas of the organism.

An embodiment of particular interest to the inventors is the use of the claimed invention in the promotion of fertility or sterility in mature plants. The inventors envision the use of the system to promote or repress the expression of genes in tissues of plants that facilitate reproduction. One of skill in the art would recognize that specific genes are known that may be selectively induced or expressed in order to regulate the fertility of a given plant. The inventors envision the transformation of plants with the Rb3 gene under the control of such promoters. Thus fertility or sterility could be controlled in such a plant through the introduction of the proper ligand.

Transgenic plants and plant cells may be transformed to contain two DNA constructs. The first chimeric construct would contain a tissue-specific promoter, an organism-specific transcription activator and an ecdysone-receptor-specific-ligand-binding domain. The second chimeric construct would contain a response element, a constitutive promoter and a Rb3 gene. Treatment of a transgenic plant containing this system with ecdysone would lead to expression of the Rb3 gene and result in infertility.

For certain uses such as hybrid production it may also be desirable to completely eliminate the male or female inflorescence. Expressing RB3 at early stages of ear or tassel development will result in failure of these organs to develop.

The present invention will be further described by reference to the following detailed examples. It is understood, however, that there are many extensions, variations, and modifications on the basic theme of the present invention beyond that shown in the examples and description, which are within the spirit and scope of the present invention. All publications, patents, and patent applications cited herein are hereby incorporated by reference.

It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions.

### EXAMPLES

### Example 1

### Isolation of Maize Rb3 Genes

Total RNA was isolated from corn tissues with TRIzol Reagent (Life Technology Inc. Gaithersburg, Md.) using a modification of the guanidine isothiocyanate/acid-phenol procedure described by Chomczynski and Sacchi [Chomczynski, P., and Sacchi, N., Anal. Biochem. 162, 156 (1987)]. In brief, plant tissue samples were pulverized in liquid nitrogen before the addition of the TRIzol Reagent, and then were further homogenized with a mortar and pestle. Addition of chloroform followed by centrifugation was conducted for separation of an aqueous phase and an organic phase. The total RNA was recovered by precipitation with isopropyl alcohol from the aqueous phase.

Poly(A)+ RNA Isolation:

The selection of poly(A)+ RNA from total RNA was performed using PolyATract system (Promega Corporation. Madison, Wis.). In brief, biotinylated oligo(dT) primers were used to hybridize to the 3′ poly(A) tails on mRNA. The hybrids were captured using streptavidin coupled to paramagnetic particles and a magnetic separation stand. The mRNA was washed at high stringent condition and eluted by RNase-free deionized water.

cDNA Library Construction:

cDNA synthesis was performed and unidirectional cDNA libraries were constructed using the SuperScript Plasmid System (Life Technology Inc. Gaithersburg, Md.). The first strand of cDNA was synthesized by priming an oligo(dT) primer containing a NotI site. The reaction was catalyzed by SuperScript Reverse Transcriptase II at 45° C. The second strand of cDNA was labeled with alpha-³²P-dCTP and a portion of the reaction was analyzed by agarose gel electrophoresis to determine cDNA sizes. cDNA molecules smaller than 500 base pairs and unligated adapters were removed by Sephacryl-S400 chromatography. The selected cDNA molecules were ligated into pSPORT1 vector between NotI and SalI sites. Zea mays tissue from tassel and vegetative meristem was employed.

Sequencing Template Preparation:

Individual colonies were picked and DNA was prepared either by PCR with M13 forward primers and M13 reverse primers, or by plasmid isolation. All the cDNA clones were sequenced using M13 reverse primers [(PROTOCOLS, Murray (ed.), pages 271-281 (Humana Press, Inc. 1991)].

### Example 2

### Using Rb3 in a Two-Hybrid System to Identify Maize Genes Involved in Control of Cell Division

The Rb3 genes and their encoded proteins can be used to identify other proteins involved in the above processes. This can be done using the Rb3 gene as bait (the target fused to the DNA-binding domain) in a yeast two-hybrid screen. Methods for two-hybrid library construction, cloning of the reporter gene, cloning of the DNA-binding and activation domain hybrid gene cassettes, yeast culture, and transformation of the yeast are all done according to well-established methods (see Sambrook et al., 1990; Ausubel et al., 1990; Hannon and Bartels, 1995). When maize Rb3 is used as bait in such a two-hybrid screen, proteins that interact with RB3 such as E2F, histone deacetylase, and chromatin-associated proteins are identified.

### Example 3

### Transient Rb3-Antisense Expression Stimulates Cell Division and Enhances Transgene Integration

A Rb3-antisense sequence is cloned into a cassette with a constitutive promoter (i.e. either a strong maize promoter such as the ubiquitin promoter including the first ubiquitin intron, or a weak constitutive promoter such as nos). Delivery of the Rb3-antisense DNA in an appropriate plant expression cassette (for example, in a UBI::ZmRb3-antisense::pinII-containing plasmid) along with UBI::bar::pinII can be accomplished through numerous well-established methods for plant transformation. Using one of these methods, DNA is introduced into maize cells capable of growth on suitable maize culture medium. Such competent cells can be from maize suspension culture, callus culture on solid medium, freshly isolated immature embryos or meristem cells. Immature embryos of the Hi-II genotype are used as the target for co-delivery of these two plasmids.

Cytological methods can be used to verify increased frequencies of progression through S-phase and mitosis (i.e. for cells in which a visual marker such as GFP was transformed alongside Rb3 the green fluorescent cells will exhibit a higher mitotic index). Cells in S-phase (undergoing DNA replication) can be monitored by detecting nucleotide analog incorporation. For example, following incubation of cells with bromodeoxyuridine (BrdU) incorporation of this thymidine analog can be detected by methods such as antiBrdU immunocytochemistry or through enhancement of Topro3 fluorescence following BrdU labeling. RB3 expression will increase the proportion of cells incorporating BrdU (i.e. a higher percentage of transformed cells will incorporate BrdU relative to untransformed cells). Increased DNA synthesis can also be monitored using such methods as fluorescence activated cell sorting (FACS) of nuclei or protoplasts, in conjunction with appropriate BrdU-insensitive fluorescent DNA labels such as propidium iodide and DAPI or BrdU-detecting methods described above. For example, tissue is homogenized to release nuclei that are analyzed using the FACS for both green fluorescence (from our accompanying GFP marker) and DNA content. Such FACS analysis demonstrates that expression of a co-transformed GFP reporter correlates with RB3-induced changes in the ratios of cells in G1, S and G2.

Similar experiments can be run using the fluorescently-labeled anti-BrdU antisera to demonstrate that RB3 expression increased the percentage of cells in S-phase. Cell cycle stage-specific probes can also be used to monitor cell cycle progression. For example, numerous spindle-associated proteins are expressed during a fairly narrow window during mitosis, and antibodies or nucleic acid probes to cyclins, histones, or DNA synthesis enzymes can be used as positive markers for the G1/S transition. For cells that have received the Rb3-antisense gene cassette, stimulation of the cell cycle is manifested in an increased mitotic index, detected by staining for mitotic figures using a DNA dye such as DAPI or Hoechst 33258. FACS analysis of Rb3-antisense-expressing cells shows that a high percentage of cells have progressed into or through S-phase. Progression through S-phase will be manifested by fewer cells in G1 and more rapid cycling times (i.e. shorter G1 and G2 stages). A higher percentage of cells are labeled when cell cycle stage-specific probes are used, as mentioned above.

To assess the effect on transgene integration, growth of bialaphos-resistant colonies on selective medium is a reliable assay. Within 1-7 days after DNA introduction, the embryos are moved onto culture medium containing 3 mg/l of the selective agent bialaphos. Embryos, and later callus, are transferred to fresh selection plates every 2 weeks. After 6-8 weeks, transformed calli are recovered. Transgenic callus containing the introduced genes can be verified using PCR and Southern blot analysis. Northern analysis can also be used to verify which calli are expressing the bar gene, and/or the Rb3-antisense construct. In immature embryos that had transient, elevated Rb3-antisense expression, higher numbers of stable transformants are recovered (likely a direct result of increased integration frequencies). Increased transgene integration frequency can also be assessed using such well-established labeling methods such as in situ hybridization.

Sometimes (i.e. using transient Rb3-antisense-mediated cell cycle stimulation to increase transient integration frequencies), it is desirable to reduce the likelihood of ectopic stable expression of Rb3-antisense. Strategies for transient-only expression can be used. This includes delivery of RNA (transcribed from the Rb3-antisense construct) along with the transgene cassettes to be integrated to enhance transgene integration by transient stimulation of cell division. Using well-established methods to produce Rb3-antisense-RNA, this can then be purified and introduced into maize cells using physical methods such as microinjection, bombardment, electroporation or silica fiber methods.

### Example 4

### Use of Antisense Oligonucleotides Against Rb3 to Transiently Stimulate Cell Division and Enhance Transgene Integration

An alternative to conventional antisense strategies is the use of antisense oligonucleotides (often with chemically-modified nucleotides). Such an antisense oligonucleotide, typically a 15-18 mer (but this size can vary either more or less), is designed to bind around accessible regions such as the ribosomal binding site around the “Start” codon. Introduction of the antisense oligonucleotide into a cell will transiently stop expression of the targeted gene. For example, an antisense oligonucleotide of between 15 to 18 nucleotides in length, that is complementary (in reverse orientation) to the sequence surrounding the Start codon of the RB3 structural gene, is introduced into maize cells. These methods of introduction for the oligonucleotide are similar to those previously described above for introduction of plasmids. Such a RB3-targeting antisense oligonucleotide will transiently depress RB3 expression, and these cells will accordingly be transiently stimulated to progress from the G1 to S phase of the cell cycle. In this manner, cell division is transiently stimulated and transgenic integration during this period is enhanced.

### Example 5

### Use of Antibodies Raised Against RB3 to Transiently Stimulate Cell Division and Enhance Transgene Integration

Genes encoding single chain antibodies directed against RB3, expressed downstream of a suitable promoter, for example the ubiquitin promoter, are introduced into maize cells using physical methods such as microinjection, bombardment, electroporation or silica fiber methods. Alternatively, single chain anti-RB3 is delivered from Agrobacterium tumefaciens into plant cells in the form of fusions to Agrobacterium virulence proteins. Fusions are constructed between the anti-RB3 single chain antibody and bacterial virulence proteins such as VirE2, VirD2, or VirF which are known to be delivered directly into plant cells. Fusions are constructed to retain both those properties of bacterial virulence proteins required to mediate delivery into plant cells and the anti-RB3 activity required for stimulating cell division and enhancing transgene integration. This method ensures a high frequency of simultaneous co-delivery of T-DNA and functional anti-RB3 protein into the same host cell.

### Example 6

### Altering RB3 Expression Stimulates the Cell Cycle and Growth

Delivery of the Rb3-antisense in an appropriate plant expression cassette is accomplished through numerous well-established methods for plant cells, including for example particle bombardment, sonication, PEG treatment or electroporation of protoplasts, electroporation of intact tissue, silica-fiber methods, microinjection or Agrobacterium-mediated transformation. As an alternative to conventional delivery of bacterial plasmids, introduction of a viral plasmid from which a Rb3-antisense sequence is expressed could also be employed.

The result of ZmRb3-antisense expression will be to stimulate the G1/S transition and hence cell division, providing the optimal cellular environment for integration of introduced genes. However, beyond the transient effects on transgenic integration, continued expression of the ZmRb3-antisense will trigger a tissue culture response (cell divisions) in genotypes that typically do not respond to conventional culture techniques, or stimulate growth of transgenic tissue beyond the normal rates observed in wild-type (nontransgenic) tissues. To demonstrate this, the Rb3-antisense gene is cloned into a cassette with a constitutive promoter (i.e. either a strong maize promoter such as the ubiquitin promoter including the first ubiquitin intron, or a weak constitutive promoter such as nos). Either particle-mediated DNA delivery or Agrobacterium-mediated delivery are used to introduce the UBI::ZmRb3antisense::pinII-containing plasmid along with a UBI::bar:pinII-containing plasmid into maize cells capable of growth on suitable maize culture medium. Such competent cells can be from maize suspension culture, callus culture on solid medium, freshly isolated immature embryos or meristem cells. Immature embryos of the Hi-II genotype are used as the target for co-delivery of these two plasmids, and within 1-7 days the embryos are transferred to culture medium containing 3 mg/l of the selective agent bialaphos. Embryos, and later callus, are transferred to fresh selection plates every 2 weeks.

After 6-8 weeks, transformed calli are recovered. In treatments where both the bar gene and Rb3-antisense gene have been transformed into immature embryos, a higher number of growing calli are recovered on the selective medium and callus growth is stimulated (relative to treatments with the bar gene alone). When the Rb3-antisense gene is introduced without any additional selective marker, transgenic calli can be identified by their ability to grow more rapidly than surrounding wild-type (nontransformed) tissues. Transgenic callus can be verified using PCR and Southern blot analysis. Northern analysis can also be used to verify which calli are expressing the bar gene, and which are expressing the maize Rb3 gene at levels above normal wild-type cells (based on hybridization of probes to freshly isolated mRNA population from the cells).

### Example 7

### Control of Rb3-Antisense Expression Using Tissue-Specific or Cell-Specific Promoters Provides a Differential Growth Advantage

A ZmRb3-antisense gene is expressed using tissue-specific or cell-specific promoters which stimulates cell cycle progression in the expressing tissues or cells. For example, using a seed-specific promoter will stimulate cell division rate and result in increased seed biomass. Alternatively, driving ZmRb3-antisense expression with a strongly-expressed, early, tassel-specific promoter will enhance development of this entire reproductive structure.

Expression of ZmRb3-antisense genes in other cell types and/or at different stages of development will similarly stimulate cell division rates. Root-specific or root-preferred expression of cyclin D will result in larger roots and faster growth (i.e. more biomass accumulation).

### Example 8

### Meristem Transformation

The Rb3-antisense sequence is cloned into a cassette with a promoter that is active within the meristem (i.e. either a strong constitutive maize promoter such as the ubiquitin promoter including the first ubiquitin intron, or a promoter active in meristematic cells such as the maize histone, cdc2 or actin promoter). Coleoptilar stage embryos are isolated and plated meristem up on a high-sucrose maturation medium [see Lowe et al.,, In Genetic Biotechnology and Breeding of Maize and Sorghum, A S Tsaftaris, ed., Royal Society of chemistry, Cambridge, UK, pp 94-97, 1997)]. The RB3-antisense expression cassette along with a reporter construct such as Ubi:GUS:pinII can then be co-delivered (preferably 24 hours after isolation) into the exposed apical dome using conventional particle gun transformation protocols. As a control the RB3-antisense construct can be replaced with an equivalent amount of pUC plasmid DNA. After a week to 10 days of culture on maturation medium the embryos can be transferred to a low-sucrose hormone-free germination medium. Leaves from developing plants can be sacrificed for GUS staining.

### Example 9

### Use of Flp/Frt System to Excise the RB3-Antisense Cassette

In cases where the Rb3-antisense has been integrated and RB3-antisense expression is useful in the recovery of maize transgenics, but is ultimately not desired in the final product, the Rb3-antisense expression cassette (or any portion thereof that is flanked by appropriate FRT recombination sequences) can be excised using FLP-mediated recombination (U.S. Pat. No. 5,929,301).

### Example 10

### Influencing Fertility

It is envisioned that the use of male (e.g. MS45) and female (e.g. nuc1 (WO 01/32856) and LEC1 (WO 00/28058) tissue-preferred promoters, could limit inducibility to specific tissues or cell types. Additionally, the EcR/USP system with tissue specific promoters could limit expression to given sights within an organism and facilitate the targeting of gene expression to just those given sights or developmental stages. The system could thus be used to selectively induce expression in only the seed or in only the reproductive structures of a plant without expression in other areas of the organism.

A tissue-preferred promoter with or without an inducible system is cloned into a plant transformation vector comprising a maize Rb3 gene. Transformation is by standard methods and regenerated plants are selected and grown. Expression of the Rb3 gene from a male tissue-preferred promoter interferes with normal cell division and fertility. Fertility is tested by analysis of pollen and fertility testing through methods known in the art. Expression of the Rb3 gene from a female tissue-preferred promoter interferes with normal cell division and fertility. Fertility is tested by analysis of seed, embryos and fertility testing through methods known in the art.

### Example 11

### Isolation of a ZmRb3 cDNA Clone From Maize Endosperm

A cDNA homologous to known Rb genes was identified in the maize EST database at Pioneer Hi-Bred Int. (Johnston, I A) by similarity to ZmRb1 in a TBLASTN search. The corresponding DNA was labeled with 32P and used to screen a ZapII cDNA library constructed from 9 DAP endosperm (Sun et al.,1997). Plaque lifting and hybridization were performed as described previously (Habben et al., 1993). The longest clone was sequenced and this sequence was re-blasted against the database at Pioneer Hi-Bred Intl. This second BLAST identified a slightly longer clone. Sequences from this clone were used to perform RACE PCR on mRNA isolated from maize kernels to find more 5′ sequence, and the new 5′ sequence was BLASTed again against the PHI/DuPont database, which turned up some orphan 5′ sequences. These clones were confirmed as Rb3 by determining the sequence of their 3′ ends. Full insert sequencing was done to confirm a novel full-length maize Rb3 gene.

### Example 12

### Transformation Methods

Maize Particle-Mediated DNA Delivery

Antisense Rb3 is cloned into a cassette with a promoter and a 3′ sequence. Particle bombardment is used to introduce the plasmid along with a UBI::PAT˜GFP::pinII plasmid into maize cells capable of growth on suitable maize culture medium. Such competent cells can be from maize suspension culture, callus culture on solid medium, freshly isolated immature embryos or meristem cells. Immature embryos of the Hi-II genotype are used as the target for co-delivery of these two plasmids. Ears are harvested at approximately 10 days post-pollination, and 1.2-1.5 mm immature embryos are isolated from the kernels, and placed scutellum-side down on maize culture medium.

The immature embryos are bombarded from 18-72 hours after being harvested from the ear. Between 6 and 18 hours prior to bombardment, the immature embryos are placed on medium with additional osmoticum (MS basal medium, Musashige and Skoog, 1962, Physiol. Plant 15:473-497, with 0.25 M sorbitol). The embryos on the high-osmotic medium are used as the bombardment target, and are left on this medium for an additional 18 hours after bombardment.

For particle bombardment, plasmid DNA (described above) is precipitated onto 1.8 μm tungsten particles using standard CaCl₂-spermidine chemistry (see, for example, Klein et al., 1987, Nature 327:70-73). Each plate is bombarded once at 600 PSI, using a DuPont Helium Gun (Lowe et al., 1995, Bio/Technol 13:677-682). For typical media formulations used for maize immature embryo isolation, callus initiation, callus proliferation and regeneration of plants, see Armstrong, C., 1994, In “The Maize Handbook”, M. Freeling and V. Walbot, eds. Springer Verlag, NY, pp 663-671.

Within 1-7 days after particle bombardment, the embryos are moved onto N6-based culture medium containing 3 mg/l of the selective agent bialaphos. Embryos, and later callus, are transferred to fresh selection plates every 2 weeks. After the first 14 days post-bombardment, the calli developing from the immature embryos are screened for GFP expression using an epifluorescent dissecting-microscope. Typically, (i.e. in the absence of a cell cycle stimulation) this is too early to observe growing multicellular transformants. Instead, as typical after such a short post-bombardment duration, numerous GFP-expressing single-cells are observed on control embryos (where the UBI::PAT˜GFP::pinII plasmid is introduced alone), but GFP-expressing multicellular clusters are not observed. It is expected that when the inventive plasmid is included along with the UBI::PAT˜GFP::pinII marker, numerous GFP+ multicellular clusters are observed growing from the immature embryos at this same early time-point (14 days post-bombardment). The higher number of rapidly-growing transformants suggests that suppression of Rb3 increases integration frequencies (thus higher numbers) and stimulates growth of these colonies after integration has occurred (thus, the transformants are clearly visible at this early juncture).

After 6-8 weeks, transformed calli are recovered. In treatments where both the PAT˜GFP gene and antisense Rb3 are transformed into immature embryos, a higher number of growing calli are expected on the selective medium and callus growth is stimulated (relative to treatments with the PAT-GFP gene alone).

Soybean Transformation

Soybean embryogenic suspension cultures are maintained in 35 ml liquid media SB196 or SB172 in 250 ml Erlenmeyer flasks on a rotary shaker, 150 rpm, 26C with cool white fluorescent lights on 16:8 hr day/night photoperiod at light intensity of 30-35 uE/m2s. Cultures are subcultured every two weeks by inoculating approximately 35 mg of tissue into 35 ml of fresh liquid media. Alternatively, cultures are initiated and maintained in 6-well Costar plates.

SB 172 media is prepared as follows: (per liter), 1 bottle Murashige and Skoog Medium (Duchefa # M 0240), 1 ml B5 vitamins 1000× stock, 1 ml 2,4-D stock (Gibco 11215-019), 60 g sucrose, 2 g MES, 0.667 g L-Asparagine anhydrous (GibcoBRL 11013-026), pH 5.7.

SB 196 media is prepared as follows: (per liter) 10 ml MS FeEDTA, 10 ml MS Sulfate, 10 ml FN-Lite Halides, 10 ml FN-Lite P, B, Mo, 1 ml B5 vitamins 1000× stock, 1 ml 2,4-D, (Gibco 11215-019), 2.83 g KNO3, 0.463 g (NH4)2SO4, 2 g MES, 1 g Asparagine Anhydrous, Powder (Gibco 11013-026), 10 g Sucrose, pH 5.8.

2,4-D stock concentration 10 mg/ml is prepared as follows: 2,4-D is solubilized in 0.1 N NaOH, filter-sterilized, and stored at −20° C.

B5 vitamins 1000× stock is prepared as follows: (per 100 ml)—store aliquots at −20° C., 10 g myo-inositol, 100 mg nicotinic acid, 100 mg pyridoxine HCl, 1 g thiamin.

Soybean embryogenic suspension cultures are transformed with various plasmids by the method of particle gun bombardment (Klein et al., 1987; Nature, 327:70.

To prepare tissue for bombardment, approximately two flasks of suspension culture tissue that has had approximately 1 to 2 weeks to recover since its most recent subculture is placed in a sterile 60×20 mm petri dish containing 1 sterile filter paper in the bottom to help absorb moisture. Tissue (i.e. suspension clusters approximately 3-5 mm in size) is spread evenly across each petri plate. Residual liquid is removed from the tissue with a pipette, or allowed to evaporate to remove excess moisture prior to bombardment. Per experiment, 4-6 plates of tissue are bombarded. Each plate is made from two flasks.

To prepare gold particles for bombardment, 30 mg gold is washed in ethanol, centrifuged and resuspended in 0.5 ml of sterile water. For each plasmid combination (treatments) to be used for bombardment, a separate micro-centrifuge tube is prepared, starting with 50 μl of the gold particles prepared above. Into each tube, the following are also added; 5 μl of plasmid DNA (at 1 μg/μl), 50 μl CaCl₂, and 20 μl 0.1 M spermidine. This mixture is agitated on a vortex shaker for 3 minutes, and then centrifuged using a microcentrifuge set at 14,000 RPM for 10 seconds. The supernatant is decanted and the gold particles with attached, precipitated DNA are washed twice with 400 μl aliquots of ethanol (with a brief centrifugation as above between each washing). The final volume of 100% ethanol per each tube is adjusted to 40 μl, and this particle/DNA suspension is kept on ice until being used for bombardment.

Immediately before applying the particle/DNA suspension, the tube is briefly dipped into a sonicator bath to disperse the particles, and then 5 UL of DNA prep is pipetted onto each flying disk and allowed to dry. The flying disk is then placed into the DuPont Biolistics PDS1000/HE. Using the DuPont Biolistic PDS1000/HE instrument for particle-mediated DNA delivery into soybean suspension clusters, the following settings are used. The membrane rupture pressure is 1100 psi. The chamber is evacuated to a vacuum of 27-28 inches of mercury. The tissue is placed approximately 3.5 inches from the retaining/stopping screen (3rd shelf from the bottom). Each plate is bombarded twice, and the tissue clusters are rearranged using a sterile spatula between shots.

Following bombardment, the tissue is re-suspended in liquid culture medium, each plate being divided between 2 flasks with fresh SB196 or SB172 media and cultured as described above. Four to seven days post-bombardment, the medium is replaced with fresh medium containing 25 mg/L hygromycin (selection media). The selection media is refreshed weekly for 4 weeks and once again at 6 weeks. Weekly replacement after 4 weeks may be necessary if cell density and media turbidity is high.

Four to eight weeks post-bombardment, green, transformed tissue may be observed growing from untransformed, necrotic embryogenic clusters. Isolated, green tissue is removed and inoculated into 6-well microtiter plates with liquid medium to generate clonally-propagated, transformed embryogenic suspension cultures.

Each embryogenic cluster is placed into one well of a Costar 6-well plate with 5 mls fresh SB196 media with 25 mg/L hygromycin. Cultures are maintained for 2-6 weeks with fresh media changes every 2 weeks. When enough tissue is available, a portion of surviving transformed clones are subcultured to a second 6-well plate as a back-up to protect against contamination.

To promote in vitro maturation, transformed embryogenic clusters are removed from liquid SB196 and placed on solid agar media, SB 166, for 2 weeks. Tissue clumps of 2-4 mm size are plated at a tissue density of 10 to 15 clusters per plate. Plates are incubated in diffuse, low light (<10 μE) at 26+/−1° C. After two weeks, clusters are subcultured to SB 103 media for 3-4 weeks.

SB 166 is prepared as follows: (per liter), 1 pkg. MS salts (Gibco/ BRL—Cat# 11117-017), 1 ml B5 vitamins 1000× stock, 60 g maltose, 750 mg MgCl2 hexahydrate, 5 g activated charcoal, pH 5.7, 2 g gelrite.

SB 103 media is prepared as follows: (per liter), 1 pkg. MS salts (Gibco/BRL—Cat# 11117-017), 1 ml B5 vitamins 1000× stock, 60 g maltose, 750 mg MgCl2 hexahydrate, pH 5.7, 2 g gelrite.

After 5-6 week maturation, individual embryos are desiccated by placing embryos into a 100×15 petri dish with a 1 cm2 portion of the SB103 media to create a chamber with enough humidity to promote partial desiccation, but not death.

Approximately 25 embryos are desiccated per plate. Plates are sealed with several layers of parafilm and again are placed in a lower light condition. The duration of the desiccation step is best determined empirically, and depends on size and quantity of embryos placed per plate. For example, small embryos or few embryos/plate require a shorter drying period, while large embryos or many embryos/plate require a longer drying period. It is best to check on the embryos after about 3 days, but proper desiccation will most likely take 5 to 7 days. Embryos will decrease in size during this process.

Desiccated embryos are planted in SB 71-1 or MSO medium where they are left to germinate under the same culture conditions described for the suspension cultures. When the plantlets have two fully-expanded trifoliate leaves, germinated and rooted embryos are transferred to sterile soil and watered with MS fertilizer. Plants are grown to maturity for seed collection and analysis. Embryogenic cultures from the CycE treatment are expected to regenerate easily. Healthy, fertile transgenic plants are grown in the greenhouse. Seed-set on CycE transgenic plants is expected to be similar to control plants, and transgenic progeny are recovered.

SB 71-1 is prepared as follows: 1 bottle Gamborg's B5 salts w/sucrose (Gibco/BRL—Cat# 21153-036), 10 g sucrose, 750 mg MgCl2 hexahydrate, pH 5.7, 2 g gelrite.

MSO media is prepared as follows: 1 pkg Murashige and Skoog salts (Gibco 11117-066), 1 ml B5 vitamins 1000× stock, 30 g sucrose, pH 5.8, 2 g Gelrite.

What is claimed is:

1. An isolated nucleic acid capable of modulating the level RB3 protein in a cell, the nucleic acid comprising a member selected from the group consisting of:
a) a polynucleotide that encodes a polypeptide comprising SEQ ID NO: 2;
b) a polynucleotide comprising the sequence set forth in SEQ ID NO: 1.
c) a polynucleotide having at least 80% sequence identity to SEQ ID NOS: 1, wherein the % sequence identity is based on the entire coding sequence of SEQ ID NO: 1 and is determined by GAP 10, using default parameters;
d) a polynucleotide fully complementary to a polynucleotide of (a) through (d).

2. The isolated nucleic acid of claim 1 adducted to a second nucleic acid sequence encoding a DNA-binding domain.

3. The isolated nucleic acid of claim 1 that is fully complementary to (c).

4. A vector comprising at least one nucleic acid of claim 1.

5. An expression cassette comprising at least one nucleic acid of claim 1 operably linked to a promoter.

6. A non-human host cell containing at least one expression cassette of claim 5.

7. The host cell of claim 6, wherein said host cell is a plant cell.

8. A transgenic plant comprising at least one nucleic of claim 1.

9. The transgenic plant of claim 8, wherein the plant is corn, soybean, sunflower, sorghum, canola, wheat, alfalfa, cofton, rice, barley, oil-seed Brassica and millet.

10. A seed produced by the plant of claim 8.

11. A seed produced by the plant of claim 9.

12. An isolated RB3 protein comprising a member selected from the group consisting of:
a) a polypeptide comprising at least 75% sequence identity to SEQ ID NO: 2, wherein the % sequence identity is based on the entire coding sequence and is determined by GAP 10 using default parameters;
b) a polypeptide encoded by a nucleic acid of claim 1; and
c) a polypeptide comprising SEQ ID NO: 2.

13. A ribonucleic acid sequence encoding a protein of claim 12.

14. A method of modulating the level of RB3 protein in a plant comprising;
a) stably transforming a plant cell with the expression cassette of claim 5; and
b) growing the transformed plant cell under plant growing conditions to produce a transformed plant.

15. The method of claim 14, wherein the plant is corn, soybean, sunflower, sorghum, canola, wheat, alfalfa, cofton, rice, barley, oil-seed Brassica and millet.

16. The method of claim 14, wherein the plant cell is selected from the group consisting of root, seed, tassel, ear, silk, stalk, embryo, flower, grain, germ, head, leaf, stem, seed, meristem and fruit.

17. A method for modulating endoreduplication comprising modulating the level of RB3 protein according to claim 14.

18. A method for modulating cell numbers in one or more tissues of a plant comprising modulating the level of RB3 protein according to claim 14.

19. A method for providing differential growth in a plant comprising modulating the level of RB3 protein according to the method of claim 14.

20. The method of claim 19, wherein the differential growth is a positive growth advantage.

21. The method of claim 14, wherein the level of RB3 protein is decreased.

22. A method for increasing crop yield, root size, plant growth, tassel size and/or ear size comprising modulating the level of RB3 protein according to the method of claim 21.

23. The method of claim 21, wherein the plant cell is quiescent cell.

24. The method of claim 14, wherein the level of RB3 protein is increased.

25. A method for conferring male sterility comprising modulating the level of RB3 protein in male reproductive tissue according to the method of claim 24.

26. A method for improving transformation frequency comprising:
a) introducing an isolated nucleic acid of claim 1 into a plant cell; and
b) transforming the plant cell with a polynucleotide of interest.

27. The method of claim 26 further comprising growing the transformed plant cell under plant growing conditions to produce a transformed plant.

28. A method for improving transformation frequency comprising:
a) introducing an isolated protein of claim 12 into a plant cell; and
b) transforming the plant cell with a polynucleotide of interest.

29. The method of claim 28 further comprising growing the transformed plant cell under plant growing conditions to produce a transformed plant.

30. A method for decreasing the level of RB3 protein in a plant cell comprising introducing into the plant cell one or more interactors that modulate RB3 protein expression in the plant cell.

31. A method for improving transformation frequency comprising decreasing the level of RB3 protein according to the method of claim 30 and transforming the plant cell with a gene of interest.

32. The method of claim 31, further comprising growing the plant cell under plant growing conditions to produce a stably transformed plant.

33. A method for modulating cell proliferation in a plant comprising:
a) stably transforming a plant cell with the expression cassette of claim 5; and
b) growing the transformed plant cell under plant growing conditions to produce a transformed plant.

34. The method of claim 33, wherein the plant cells are involved in floral development, organ formation or branch/tiller initiation.

35. The method of claim 33, wherein the plant cells are involved in fertility.

36. The method of claim 33, wherein cell proliferation is inhibited.

37. A method for identifying RB3 interacting proteins comprising adducting the nucleic acid sequence of claim 1 to a second nucleic acid sequence encoding a DNA-binding domain.
